(19) United States
(12) Patent Application Publication
(10) Pub. No.: US 2020/0197513 A1
(43) Pub. Date: Jun. 25, 2020

(54) NUTRACEUTICAL BLENDS

(71) Applicant: KAIROS BIOMEDICAL, INC., Kailua, HI (US)

(72) Inventor: A. William SCHWARTZ, Honolulu, HI (US)

(21) Appl. No.: 16/612,637

(22) PCT Filed: May 10, 2018
(86) PCT No.: PCT/US2018/032150

§ 371 Date: Nov. 11, 2019

Related U.S. Application Data
(60) Provisional application No. 62/505,641, filed on May 12, 2017.
(60) Provisional application No. 62/663,986, filed on Apr. 27, 2018.

(51) Int. Cl. A61K 39/39; A61K 36/06; A61K 35/745; A61P 35/00; A61K 9/00

(52) U.S. Cl. CPC A61K 39/39; A61K 36/06; A61K 2039/585; A61P 35/00; A61K 9/0053; A61K 35/745

(57) ABSTRACT

Compositions, formulations and kits of combinations of prebiotic and probiotic components are disclosed. The combinations may be useful for enhancing immune system functions and enhancing immune response to cancer in a subject.

### PRIORITY AND CROSS-REFERENCE TO RELATED APPLICATIONS

This application claims the benefit of U.S. Provisional Application 62/505,641, filed on May 12, 2017, and U.S. Provisional Application 62/663,986, filed on Apr. 27, 2018, which are hereby incorporated by reference in its entireties.

### BACKGROUND

### Field

The present disclosure relates nutraceutical blends for improving immune system function. Some embodiments of the present disclosure relate to combinations of prebiotic and probiotic compounds, and compositions and/or kits thereof, for improving immune system functions.

### Description of the Related Art

Probiotics are microorganisms that are believed to provide some health benefits when consumed. They may be ingested by humans, animals or both. Prebiotics are non-digestible food ingredients that stimulate and/or enhance the growth and/or activity of bacteria in the digestive system in ways that are beneficial to health.

One prebiotic that has been used as a nutritional supplement is Active Hexose Correlated Compound, which is a bioactive fungal extract (comprising a mixture of several chemicals including at least proteins, fats, carbohydrates and glucans), produced from the mycelia of shiitake (Lentinula edodes) of the basidiomycete family of mushrooms. Active Hexose Correlated Compound is referred to generically in this application as “AHCC”, which simply represents the acronym for this extract, and as used herein does not refer to any particular brand name AHCC product on the market.

### SUMMARY

A combination is disclosed for activating immune cells and/or inducing cytokines. The combination comprises: a bioactive fungal extract derived from one or more different strains of Lentinula edodes, wherein the bioactive fungal extract comprises about 60%-90% by weight carbohydrates, comprising polysaccharides including α(1-4) glucans and β(1-3) glucans; and a probiotic component, wherein the bioactive fungal extract and the probiotic component interact synergistically to activate immune cells and/or induce cytokines.

In one embodiment of the combination, the bioactive fungal extract is a commercially available mushroom extract referred to as Active Hexose Correlated Compound (hereinafter referred to by the generic acronym “AHCC”).

In another embodiment of the combination, the bioactive fungal extract is K1.

In one embodiment of the combination, the probiotic component is selected from the group consisting of Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

In one variation, the probiotic component is B. lactis or metabolites and/or probiotic cell wall fractions derived from cultures of B. lactis.

In another variation to the combination, the bioactive fungal extract and the probiotic component are formulated for oral delivery. The oral delivery formulation may include both the bioactive fungal extract and the probiotic component together in a single unit dosage form.

In one embodiment, the combination of the bioactive fungal extract and the probiotic component are disclosed for use in treating and/or preventing cancer.

In one embodiment, the combination of the bioactive fungal extract and the probiotic component are disclosed for use in treating and/or preventing autoimmune diseases, allergies and inflammation.

A method for activating immune cells in accordance with one embodiment. The method comprises: administering to the immune cells a combination comprising an effective amount of a bioactive fungal extract and an effective amount of a probiotic component, wherein the effective amounts are sufficient to synergistically activate the immune cells.

In a variation to the method, the bioactive fungal extract is AHCC.

In another variation to the method, the bioactive fungal extract is K1.

In another variation to the method, the probiotic component is selected from the group consisting of Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

In one particular embodiment of the method, the probiotic component is B. lactis or metabolites and/or probiotic cell wall fractions derived from cultures of B. lactis.

In one embodiment of the method, the bioactive fungal extract and a probiotic component in the combination may be administered simultaneously.

In one embodiment of the method, the bioactive fungal extract and a probiotic component in the combination are administered sequentially.

In one embodiment of the method, the immune cell activation includes upregulating expression of CD69 on lymphocytes and monocytes. In another embodiment, the immune cell activation includes activation of one or more of NK cells, monocytes and macrophages. In yet another embodiment, the immune cell activation includes activation of anti-inflammatory cytokines IL-1ra and IL-10.

A method is disclosed for reducing viability of cancer cells. The method comprises: administering to the cancer cells a combination comprising an effective amount of a bioactive fungal extract and an effective amount of a probiotic component, wherein the effective amounts are sufficient to synergistically reduce the viability of the cancer cells.

In one embodiment of the method for reducing viability of cancer cells, the bioactive fungal extract is AHCC. In one embodiment of the method for reducing viability of cancer cells, the bioactive fungal extract is K1. In another embodiment, the probiotic component is selected from the group consisting of Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

In a further embodiment of the method for reducing viability of cancer cells, the probiotic component is B. lactis or metabolites and/or probiotic cell wall fractions derived from cultures of B. lactis.

The bioactive fungal extract and a probiotic component in the combination may be administered simultaneously, or they may be administered sequentially.

In one embodiment, a kit is disclosed for activating immune cell function. The kit comprises: a combination of an effective amount of a bioactive fungal extract, and an effective amount of a probiotic; and instructional materials for using the combination to treat or prevent a condition for which activating immune cell function may be therapeutic or prophylatic; wherein the amounts of bioactive fungal extract and probiotic are effective in combination to synergistically active immune cell function.

In one variation, the bioactive fungal extract in the kit is AHCC.

In one variation, the bioactive fungal extract in the kit is K1.

In another variation to the kit, the synergistic activation of immune cell function comprises one or more of the following: upregulating expression of CD69 on lymphocytes and monocytes; activation of one or more of NK cells, monocytes and macrophages; activation of one or more of anti-inflammatory cytokines IL-1ra and IL-10; activation of one or more of IL-8, G-CSF and TNF-alpha; and synergistic reduction cancer cell viability.

### BRIEF DESCRIPTION OF THE DRAWINGS

The following detailed description of preferred embodiments of the disclosure will be better understood when read in conjunction with the appended drawings. For the purpose of illustrating the disclosure, there are shown in the drawings embodiments which are presently preferred. It should be understood, however, that the disclosure is not limited to the precise arrangements and instrumentalities of the embodiments shown in the drawings.

FIG. 1 depicts the results of an experiment comparing hematocrit levels.

FIG. 2 depicts the results of an experiment comparing the number of CD11c+ myeloid dendritic cells.

FIG. 3 depicts the results of an experiment comparing the number of monocyte cells.

FIG. 4 depicts the results of an experiment comparing the number of CD14+CD16+ sub set of monocyte cells.

FIG. 5 depicts the results of an experiment comparing the level of vaccine-specific IgG3.

FIG. 6 shows an embodiment of a schematic of an experimental schedule of investigating the effect of embodiments of AHCCs of the present disclosure on murine vaginal Candidiasis.

FIG. 7 shows microscopic images of hyphal candida cells as pathogenic growth form from vaginal cavities.

FIG. 8 shows data related to the effect of oral administration of prebiotics and probiotics of the present disclosure on the number of the viable C. albicans cells in the vaginal cavity.

FIG. 9 shows data related to the effect of oral administration of prebiotics and probiotics of the present disclosure on C. albicans cells in the vaginal cavities of mice with vaginal candidiasis.

FIG. 10 shows data related to the effect of administration of prebiotics and probiotics of the present disclosure on CD14+CD16+ monocytes of human volunteers.

FIG. 11 shows flow cytometry data with gates for lymphocytes, monocytes, and the four subsets of lymphocytes, allowing analysis of CD69 expression on all five cell types.

FIG. 12 shows the effects K1 and AHCC on NK cell activation. The percent change in CD69 expression from the untreated control cultures are shown as the average±standard deviation for each data set.

FIG. 13 shows the effects K1 and AHCC on anti-inflammatory cytokines. The percent change in cytokine levels from the untreated control cultures are shown as the average±standard deviation for each data set.

FIG. 14 shows the effects K1 and AHCC on immune-activating pro-inflammatory cytokines and chemokines in human cell cultures. activation. The percent change in cytokine levels from the untreated control cultures are shown as the average±standard.

FIG. 15 shows the effect of K1 and AHCC (1 mg/mL) on the MCF-7 breast cancer cell line is shown as a result of the conversion of MTT reagent to the colored compound Formazan by the mitochondrial reductase enzyme in viable cells. K1 triggered reduced cell viability, as reflected by a reduced colorimetric reaction in the MTT assay. This effect was highly significant when compared to untreated cultures and AHCC-treated cultures.

FIG. 16 shows the effects K1 (0.2 mg/mL), the probiotic cell wall fraction (PCW), and the blend thereof (K1 PCW) on NK cell activation. The mean fluorescence intensity of CD69 expression are shown as the average±standard deviation for each data set.

FIG. 17 shows the effects K1 and AHCC, alone and blended with probiotic metabolites (PMET) versus probiotic cell walls (PCW) on the immune-activating pro-inflammatory cytokines TNF-a and IL-8, the anti-inflammatory cytokine IL-10, and the stem cell growth factor G-CSF in human immune cell cultures. The percent change in cytokine levels from the untreated control cultures are shown as the average±standard deviation for each data set. Statistical significance levels are indicated for the comparison of K1 alone versus blended with probiotic fractions, where P<0.05 is indicated by a single asterisk *, and a high level of significance P<0.01 is indicated by a double asterisk **.

FIG. 18 shows the effect of K1 and AHCC (1 mg/mL) on the MCF-7 breast cancer cell line is compared to the effects of probiotic cell walls (PCW), as well as blends of K1/PCW and AHCC/PCW. The data reflect the conversion of MTT reagent to the colored compound Formazan by the mitochondrial reductase enzyme in viable cells. Both K1 and PCW triggered reduced cell viability, as reflected by a reduced colorimetric reaction in the MTT assay, when compared to untreated control cultures. The K1/PCW blend triggered more robust reduction of MCF-7 viability than either ingredient alone. The difference between K1 or PCW alone, versus the K1/PCW blend was highly significant when compared to untreated cultures and AHCC-treated cultures (P<0.01, **).

### DETAILED DESCRIPTION

There is an enormous body of evidence that supports the notion that many chronic diseases can be prevented, or at least substantially delayed. Thousands of phytochemicals are known to protect against disease. Often grouped with these are medicinal fungi with medicinal mushrooms being among the examples of foods that contain disease-mitigating ingredients. The practice of using mushrooms as “medical foods” is found in the traditions of many cultures. Without being limited by any particular theory, it could be envisioned that the early herbalist was probably more interested in the medicinal properties of mushrooms than their use as food.

The present disclosure is related to prebiotic and probiotic compounds, and compositions and/or kits thereof, for improving and/or augmenting immune cell function. In some embodiments, the present disclosure is related to combinations of prebiotic and probiotic compounds, and compositions and/or kits thereof, for improving and/or augmenting immune cell function. In some embodiments, the prebiotic compounds and probiotic compounds exhibit synergistic interactions for improving and/or augmenting immune cell function.

The present disclosure also relates to methods of prevention and/or treatment of one or more diseases by providing combinations of prebiotic and probiotic compounds, and the compositions and/or kits thereof, disclosed herein to a subject in need thereof. In some embodiments, the present disclosure relates to methods of enhancing a subject's immune system by providing combinations of prebiotic and probiotic compounds, and the compositions and/or kits thereof, disclosed herein. In some embodiments, the present disclosure relates to methods of enhancing a subject's immune response to vaccination by providing combinations of prebiotic and probiotic compounds, and the compositions and/or kits thereof, disclosed herein.

### Definitions

It is to be understood that the figures and descriptions of the present disclosure have been simplified to illustrate elements that are relevant for a clear understanding of the present disclosure, while eliminating, for the purpose of clarity, many other elements found in typical microscope devices. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The phrase “biological sample” is used herein in its broadest sense. A sample may be of any biological tissue or fluid from which biomarkers of the present disclosure may be detected, extracted, isolated, characterized or measured. Examples of such samples include but are not limited to blood, lymph, urine, gynecological fluids, biopsies, amniotic fluid and smears. Samples that are liquid in nature are referred to herein as “bodily fluids.” Biological samples may be obtained from a patient by a variety of techniques including, for example, by scraping or swabbing an area or by using a needle to aspirate bodily fluids. Methods for collecting various biological samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological samples also include tissues, such as, frozen sections taken for histological purposes. The sample also encompasses any material derived by processing a biological sample. Derived materials include, but are not limited to, cells (or their progeny) isolated from the sample, proteins or nucleic acid molecules extracted from the sample. Processing of a biological sample may involve one or more of filtration, distillation, extraction, concentration, inactivation of interfering components, addition of reagents, and the like.

The term “cancer” as used herein is defined as a hyperproliferation of cells whose unique trait—loss of normal control—results in unregulated growth, lack of differentiation, local tissue invasion, and/or metastasis. Examples include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, germ-cell tumors, and the like.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition.

As used herein, the term “container” includes any receptacle for holding the pharmaceutical composition. For example, in some embodiments, the container is the packaging that contains the pharmaceutical composition. In other embodiments, the container is not the packaging that contains the pharmaceutical composition, i.e., the container is a receptacle, such as a ⋅ box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity or frequency of at least one sign or symptom of the disease or disorder experienced by a patient is reduced.

As used herein “endogenous” refers to any material from or produced inside an organism, cell, tissue or system.

As used herein, the term “exogenous” refers to any material introduced from or produced outside an organism, cell, tissue or system.

“Instructional material,” as that term is used herein, includes a publication, a recording, a diagram, or any other medium of expression, which can be used to communicate the usefulness of components of the disclosure in a kit for activating immune cell function, identifying or alleviating or treating various diseases or disorders disclosed herein. Optionally, or alternately, the instructional material may describe one or more methods of identifying or alleviating the diseases or disorders in a cell or a tissue of a subject. The instructional material of the kit may, for example, be affixed to a container that contains the compositions of the disclosure or be shipped together with a container that contains the compositions of the disclosure. Alternatively, the instructional material may be shipped separately from the container with the intention that the recipient uses the instructional material and the compound cooperatively.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject.

The term encompasses perturbing and/or affecting a native signal or response thereby mediating a beneficial therapeutic response in a subject, preferably, a human.

As used herein, “nutraceutical blend,” “nutritional combination,” “nutritional composition,” “nutritional formulation,” or “nutritional formula” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. The term “patient” as used herein is meant to include a human or a veterinary patient. Within the context of the present disclosure, veterinary patients include both mammalian and non-mammalian veterinary patients, the latter including such veterinary patients as, for example, lizards and birds.

The terms “prebiotic,” “prebiotic component,” “prebiotic compound” and the like include any substance or combination of substances that may be utilized as a nutrient by a microorganism, may induce the growth and/or activity of a microorganism, may induce the replication of a microorganism, may be utilized as an energy source by the microorganism, and/or may be utilized by the microorganism for the production of biomolecules (i.e. RNA, DNA, and proteins). Non-limiting examples of prebiotics include mucopolysaccharides, oligosaccharides, polysaccharides, amino acids, vitamins, nutrient precursors, harvested metabolic products of biological organisms, microbial lysates, lipids, and proteins.

The terms “probiotic,” “probiotic organism,” “probiotic component,” “probiotic compound” and the like include live microorganisms, as well as cell fractions of microorganisms, such as metabolites and cell wall fractions, that beneficially affect the health of a host. The benefits to the health of the host include, but are not limited to, improving the microbial balance of the intestines. Other beneficial effects to the host include, for example, enhancing the immune system, stimulation of phagocytotic activity, stimulation of interferon, reduction of hypertension, decrease in the risk of cancer, increase in antimicrobial activity and immunomodulating effects, reduction of hypercholesterolemia, and treatment of cancer.

As used herein, the term “pharmaceutical composition” refers to a mixture of at least one compound of the disclosure with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, intramuscular, oral, aerosol, parenteral, ophthalmic, pulmonary and topical administration. “Administering” as used herein may mean conventional delivery to a human or non-human subject, via any of the above-listed techniques. In some embodiments, administering may be delivering the disclosed compounds/combinations directly to cells in vitro. In some embodiments, administering may mean conventional delivery to a human or non-human subject, including delivering the disclosed compounds/combinations to target cells in the subject, e.g., immune cells for which immune activation is desired.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference

As used herein, “immune cells” refer to, without limitations, natural killer cells, mast cells, eosinophils, basophils, phagocytic cells including macrophages, neutrophils, and dendritic cells, antigen presenting cells, naïve T cells, naïve B cells, effector T cells, effector B cells, memory T cells, memory B cells, helper T cells, regulatory T cells, plasma cells, megakaryocytes, PBMCs, and other cells of the innate and adaptive immune systems that function by identifying and eliminating pathogens that might cause infection.

“An antigen presenting cell” (APC) is a cell that are capable of activating T cells, and includes, but is not limited to, monocytes, macrophages, B cells, and dendritic cells (DCs).

The term “B cell” as used herein is defined as a cell derived from the bone marrow and/or spleen. B cells can develop into plasma cells which produce antibodies.

The term “T cell” as used herein is defined as a thymus-derived cell that participates in a variety of cell-mediated immune reactions.

The term “T-helper” as used herein with reference to cells indicates a subgroup of lymphocytes (a type of white blood cell or leukocyte) including different cell types identifiable by a skilled person. In particular, T-helper cell according to the present disclosure include effector Th cells (such as Th1, Th2 and Th17). These Th cells secrete cytokines, proteins or peptides that stimulate or interact with other leukocytes.

As used herein, “immune activation” refers to morphological and/or phenotypic changes in one or more immune cells including, but not limited to, increased and/or decreased proliferation, increased and/or decreased cytokine production, increased and/or decreased expression of cell surface markers, increased and/or decreased size, and combinations thereof.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e. arresting its development: or (c) relieving the disease, i.e. causing regression of the disease.

The term “therapeutic” as used herein means a treatment and/or prophylaxis. A therapeutic effect is obtained by suppression, remission, or eradication of a disease state.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

In some embodiments, an effective amount of each of two compounds in a combination, or more particularly, a prebiotic and probiotic combination (e.g., AHCC and B. lactis) is a synergistic amount—e.g., an amount of each component that provide a synergistic effect of the combination compared to the additive effects of both components administered as a monotherapy. As used herein, a “synergistic combination” or a “synergistic amount” is a combination or amount that is more effective in the therapeutic or prophylactic treatment of a disease than the incremental improvement in treatment outcome that could be predicted or expected from a merely additive combination of the therapeutic or prophylactic benefit of the individual components of the combination when administered at that same dosage as a monotherapy. In some embodiments, the “synergistic combination” or a “synergistic amount” is a combination or amount that is more effective in activating immune cell function than the incremental improvement in activation that could be predicted or expected from a merely additive combination of the immune cell activating effect(s) of the individual components of the combination when administered at that same dosage as a monotherapy.

Throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

An additive effect is observed when the potentiation is equal to the sum of the individual effects of the anti-cancer agent(s) and modulator(s). A synergistic effect is observed when the potentiation is greater than the sum of the individual effects of the anti-cancer agent and modulator(s). A synergistic effect is greater than an additive effect. Synergistic effect, additive effect or both can occur in human patients, non-human patients, non-patient human volunteers, in vivo models, ex vivo models, in vitro models, etc.

Potentiation can range from about <1 to about 100-fold. In some embodiments, the synergistic effect is about 3 to about 30-fold. In some embodiments, the potentiation ranges from <1, 1, >1, 2, 3, 4, 5, 6, 7, 8, 9, 10, 20, 30, 40, 50, 60, 70, 80, 90, or 100 fold, or within a range defined by any two of the aforementioned values.

### Prebiotic

In various embodiments, a prebiotic is a compound that is difficult to digest, non-digestible or essentially non-digestible by a human. In some embodiments, a prebiotic acts to promote the growth of one or more probiotics.

In some embodiments, a prebiotic can be one or more saccharides that are non-digestible by the human host and can act as a non-digestible fiber in the diet. This non-digestibility is because humans lack the enzymes to break down some or all of the prebiotic saccharides as they travel through the digestive tract. When a prebiotic reaches the small intestine and colon, bacteria encoding an enzyme or enzymes capable of digesting the prebiotic can break down the prebiotic into simple sugars that the bacteria can use.

Without limitations, prebiotics can comprise one or more of a carbohydrate, carbohydrate monomer, carbohydrate oligomer, or carbohydrate polymer. In some embodiments, the prebiotic is a fungal extract. In some embodiments, the fungal extract is AHCC. In other embodiments, the fungal extract is K1.

### Active Hexose Correlated Compound

Active Hexose Correlated Compound, or “AHCC” is a commercially available medicinal mushroom extract product. AHCC is an alpha-glucan-rich dietary supplement extracted from the mycelia of Basidiomycota mushrooms, such as shitake (Latninula edodes). It contains a mix of oligosaccharides (comprising around 74% of AHCC, approximately 20% of which is the alpha-1,4-glucantype), amino acids, lipids, and minerals. AHCC is water soluble, water stable, and may be microcoated with candelilla wax in some embodiments to improve solubility in the intestines. AHCC was originally developed in Japan in 1992, with several studies reporting a variety of therapeutic effects, including antioxidant and anticancer activity when combined with one or more other compounds, prevention of diabetes onset and liver injury, and improvement of immune response.

In Japan, various mushrooms and tree fungi have a long history of medicinal use. AHCC is made from the mycelia (vegetative portion) of various mushrooms in the general family of basidiomycete. It has been used to treat cancer with some apparent success.

In some embodiments, AHCC is a mixture of polysaccharides, amino acids, lipids and minerals derived from cocultured mycelia of several species of Basidiomycete mushrooms. AHCC has been implicated with immunomodulation and protection against infection. AHCC can enhance tumor immune surveillance by regulating both innate and adaptive immune responses (Gao, Y. et al., Cancer Immunol. Immunother., 55(10): 1258-1266, 2006; Ritz, B. W. et al., J. Nutr. 136:2868-2873, 2006, which are hereby incorporated by reference in their entireties). Several human clinical studies have shown the potential beneficial effects of AHCC in being generally safe, preventing and/or treating cancer, mitigating the side effects of cancer chemotherapy, improving liver function, and improving immune function in general and against vaccines (See, ahccresearch.com, which is hereby incorporated by reference in its entirety).

Many acute and chronic conditions are caused by compromised immunity and AHCC (acting as a biological response modifier) augments natural immune responses (See, ahccresearch.com, which is hereby incorporated by reference in its entirety). AHCC has been used successfully to treat a wide range of health conditions such as colds, flu, cancer, hepatitis, diabetes, and cardiovascular diseases (See, ahccresearch.com, which is hereby incorporated by reference in its entirety).

Clinical trials have shown that AHCC improves both innate and adaptive immune responses by increasing cytokine production, increasing NK cell activity, increasing macrophage populations, increasing dendritic cell numbers, and increasing T cell numbers. (See, ahccresearch.com, which is hereby incorporated by reference in its entirety).

For example, as shown in Example 3, an increase in CD14+ CD16+ monocytes, cells that are involved in phagocytosis, antigen-presentation, cytokine production, and immune regulation, was observed in smokers who were administered an embodiment of a composition according to the present disclosure.

Thus, in some embodiments, AHCC may increase macrophage antigen presentation activity and inhibition of tumor-derived immune suppressive factors, enhance macrophage proliferation and activation, promote differentiation of Th1 cells; increase macrophage production of IL-12, increase NK activity; promote apoptosis of cancer cells. AHCC in cancer patients has been reported to increase TNF-a, y-interferon, interleukin-12 and decrease immunosuppressive acidic protein (IAP) and tumor growth factor (TGF)-a.

In some embodiments, AHCC is a mixture of several macromolecules. In some embodiments, AHCC is a mixture of protein, fat, dietary fiber, glucans, ash, and carbohydrate. In some embodiments, AHCC comprises alpha glucans. In some embodiments, AHCC comprises acetylated α glucans. In some embodiments, AHCC comprises acetylated α 1,4 glucans. In some embodiments, AHCC comprises low levels of beta glucans.

In some embodiments, AHCC comprises any of the macromolecules in any amount described herein. In some embodiments, the protein content in AHCC mixture ranges from about 2.5% to about 65%. In some embodiments, the fat content in AHCC mixture ranges from about 0.4% to about 12%. In some embodiments, the dietary fiber in AHCC mixture ranges from about 0.4% to about 12%. In some embodiments, the total glucan content in AHCC mixture is about 2%. In some embodiments, the beta glucan content in AHCC mixture is very low, ranging from about 0.04% to about 0.2%. In some embodiments, the ash content in AHCC mixture ranges from about 1.5% to about 45%. In some embodiments, the carbohydrate content in AHCC mixture ranges from about 14% to about 75%. In some embodiments, the protein content in AHCC mixture is about 13%. In some embodiments, the fat content in AHCC mixture is about 2.5%. In some embodiments, the dietary fiber in AHCC mixture is about 2%. In some embodiments, the ash content in AHCC mixture is about 9%. In some embodiment, the carbohydrate content in AHCC mixture is about 70%.

In some embodiments, the molecular weight of AHCC mixture ranges from about 500 Da to about 50,000 Da. In some embodiments, the molecular weight of AHCC ranges from about 1000 Da to about 10,000 Da. In some embodiments, the molecular weight of AHCC is less than about 5000 Da. In some embodiments, the molecular weight of AHCC is more than about 5000 Da. In some embodiments, the molecular weight of AHCC is about 5000 Da.

In some embodiments, a molecular weight of AHCC of about 5000 Da makes AHCC much more bioavailable and effective in stimulating the immune system. In some embodiments, a molecular weight of AHCC of about 5000 Da allows the body to break down AHCC into smaller fractions with lower molecular weights.

In some embodiments, AHCC is produced using one or more methods known in the art. In some embodiments, the method of production uses one or more of mushrooms. In some embodiments, the type of mushroom used includes without limitation, Lentinula aciculospora, Lentinula boryana, Lentinula edodes, Lentinula guarapiensis, Lentinula lateritia, Lentinula novae-zelandiae, Lentinula raphanica, Lentinula reticeps, Trametes gibbosa—Lumpy bracket, Trametes hirsuta—Hairy bracket, Trametes nivosa, Trametes pubescens, Trametes versicolor—Turkey tail, Trametes (Coriolus), and species of the Schizophyllum genus.

In some embodiments, fermentation and extraction duration ranges from about 21 days to about 84 days. In some embodiments, fermentation and extraction duration ranges from about 14 days to about 91 days. In some embodiments, fermentation and extraction duration ranges from about 45 days to about 60 days.

### K1

K1 is a novel fungal compound produced by cultivating a mixture of Lentinula edodes cultivars on a solid substrate of hydrated cooked grains for about 10-100 days, as disclosed in greater detail in co-pending U.S. Provisional Application No. 62/663,878, which is incorporated herein in its entirety by reference thereto. K1 is a bioactive fungal product. In some embodiments, K1 is a dried bioactive fungal product. In some embodiments, K1 is produced from one or more mushroom mycelium cultures. In some embodiments, K1 is produced from one or more mushroom mycelium cultures comprising one or more strains of Lentinula edodes.

A unique bioactive compound, designated K1, was extracted from a solid substrate that had been inoculated with filamentous fungi and incubated for an extended time. The compound ranged from 20% to 30% of starting dry weight after concentration and the full ¹H NMR spectra showed a complex mixture of aromatic, polysaccharides/sugars, and aliphatic amino, lipid and organic acid components. The bioactive fungal extract K1 exhibited strong immune activating properties. In some embodiments, K1 is produced using a solid substrate comprising hydrated cooked rice, wheat or rye. In some embodiments, K1 is produced by a method comprising an incubation period of about 60 days.

In some embodiments, K1 comprises alpha glucans. In some embodiments, K1 comprises acetylated α glucans. In some embodiments, K1 comprises acetylated α 1,4 glucans In addition, K1 also contains beta-glucans. In some embodiments, the amount of α glucans ranges from about 9.9% w/w to about 25.0% w/w. In some embodiments, the amount of beta glucans ranges from about 10.9% w/w to about 30.2% w/w. In some embodiments, the amount of α glucans ranges from about 5% w/w to about 50% w/w. In some embodiments, the amount of beta glucans ranges from about 5.5% w/w to about 60% w/w.

In some embodiments, K1 is characterized by the following properties: about 81.5% by weight carbohydrates; about 3.4% by weight protein; about 0.7% lipid by weight; about 14.4% by weight of other components; and immunomodulatory bioactivity.

In some embodiments, K1 comprises about 75% to about 85% by weight carbohydrates. In some embodiments, K1 comprises about 2.5% to about 5% by weight proteins. In some embodiments, K1 comprises about 0.5% to about 1.5% by weight lipids. In some embodiments, other components comprise about 8.5% to about 22% by weight of K1.

In some embodiments, K1 is characterized by HPLC. In some embodiments, K1 is characterized by HPLC of a de-lipidized dried product sample run on an Agilent Hi-Plex Na column. In some embodiments, K1 exhibits three retention time peaks by HPLC on Agilent Hi-Plex Na column. In some embodiments, K1 exhibits three retention time peaks by HPLC on Agilent Hi-Plex Na column at about 10.5, 11.2, and 24.0. In some embodiments, the column is eluted with water at 0.3 ml/min at 85° C. and under a pressure of about 25 bar. In some embodiments, the column eluate is detected by UV at 280 nm.

In some embodiments, K1 is characterized as comprising about 60% to about 70% by weight carbohydrates. In some embodiments, K1 is characterized as comprising about 3.5% to about 5.5% by weight proteins. In some embodiments, K1 is characterized as comprising about 1.5% to about 4.5% by weight lipids. In some embodiments, other components comprise about 20% to about 35% by weight of K1.

In some embodiments, the one or more dried products comprising bioactive fungal compounds are characterized by the following properties: 60%-90% by weight carbohydrates; 3%-5% by weight protein; 0.1%-3% by weight lipid; 2 or 3 retention time peaks by HPLC; and immunomodulatory bioactivity.

In some embodiments, the carbohydrates in the dried product comprises polysaccharides. In some embodiments, the polysaccharides comprise α(1-4) glucans and β(1-3) glucans as determined by 1H spectrum. In some embodiments, the polysaccharides comprise α(1-4) glucans and β(1-3) glucans as determined by 13C NMR spectrum. In some embodiments, the polysaccharides comprise α(1-4) glucans and β(1-3) glucans as determined by 1H or 13C NMR spectrum. In some embodiments, the polysaccharides comprise α(1-4) glucans and β(1-3) glucans as determined by 1H and 13C NMR spectrum. In some embodiments, the polysaccharides have a median molecular weight of about 10,000 Da. In some embodiments, the polysaccharides have a median molecular weight of about 7,500 Da to about 12,500 Da. In some embodiments, the polysaccharides have a median molecular weight of about 7,500, 8,000, 8,500, 9,000, 9,500, 10,000, 10,500, 11,000, 11,500, 12,000, or 12,500 Da, or a value within a range defined by any two of the aforementioned values.

In some embodiments, the dried product comprises about 3% to about 5% protein by weight. In some embodiments, the dried product comprises 0.5, 1, 1.5, 2, 2.5, 3, 3.5, 4, 4.5, 5, 5.5, 6, 6.5, 7, 7.5, 8, 8.5, 9, 9.5, or 10% protein by weight, or a value within a range defined by any two of the aforementioned values.

In some embodiments, the dried product comprises about 0.1% to about 3% lipid by weight. In some embodiments, the dried product comprises 0.0125, 0.025, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, or 6% lipid by weight, or a value within a range defined by any two of the aforementioned values.

In some embodiments, the dried product yields two retention time peaks by HPLC of de-lipidized sample on Agilent Hi-Plex Na column. In some embodiments, the dried product yields three retention time peaks by HPLC of de-lipidized sample on Agilent Hi-Plex Na column. In some embodiments, the dried product yields two or three retention time peaks by HPLC of de-lipidized sample on Agilent Hi-Plex Na column.

In some embodiments, the dried product exhibit biological activities. In some embodiments, the dried product exhibits immunomodulatory bioactivity. Non-limiting examples include anti-viral activity, immune cell activation, induction of cytokines, induction of chemokines, and/or induction of growth factors. In some embodiments, the biological activities of the dried extract are determined. The biological activities of the powdered extract may be determined by any conventional assays such as FACS, ELISA, ELISPOT, Western blotting, immunoassays, cell-based in vitro assays and animal-based in vivo assays, etc.

In some embodiments, the immunomodulatory bioactivity comprises activation of one or more cells selected from NK cells, B-lymphocytes, dendritic cells, stem cells or monocytes. In some embodiments, the immunomodulatory bioactivity comprises at least one of activation of one or more immune cells selected from NK cells, NKT cells, T-lymphocytes, Non-T and Non-NK lymphocytes, or monocytes, activation of pro-inflammatory cytokines/chemokines, activation of anti-inflammatory cytokines/chemokines, and activation of both pro- and anti-inflammatory cytokines/chemokines.

In some embodiments, K1 exhibits biological activities. In some embodiments, K1 exhibits immunomodulatory bioactivity. Non-limiting examples include anti-viral activity, immune cell activation, induction of cytokines, induction of chemokines, and/or induction of growth factors. In some embodiments, K1 exhibits immunomodulatory bioactivity. In some embodiments, the immunomodulatory bioactivity comprises at least one of activation of one or more immune cells selected from NK cells, NKT cells, T-lymphocytes, Non-T and Non-NK lymphocytes, or monocytes, activation of pro-inflammatory cytokines/chemokines, activation of anti-inflammatory cytokines/chemokines, and activation of both pro- and anti-inflammatory cytokines/chemokines.

In some embodiments, the dried product according to the present disclosure may be useful in preventing and/or treating diseases. Non-limiting examples, include cancer, immune-related diseases such as autoimmune diseases, allergies and inflammation, and infectious diseases, such as influenza, common cold and respiratory illnesses.

### Probiotics

Bifidobacterium is a type of gram-positive, non-motile anaerobic bacteria. It is an endosymbiotic inhabitant within the body, such as in the gastrointestinal tract of mammals, including humans. Bifidobacterium are one of the major genera of bacteria that make up the colon flora in mammals. Some Bifidobacterium strains are considered as important probiotics and used in the food industry. Different species and/or strains of Bifidobacterium may exert a range of beneficial health effects, including the regulation of intestinal microbial homeostasis, the inhibition of pathogens and harmful bacteria that colonize and/or infect the gut mucosa, the modulation of local and systemic immune responses, the repression of procarcinogenic enzymatic activities within the microbiota, the production of vitamins, and the bioconversion of a number of dietary compounds into bioactive molecules. It is believed that Bifidobacterium improve the gut mucosal barrier and lowers levels of lipopolysaccharide in the intestine.

The genus Bifidobacterium possesses a unique fructose-6-phosphate phosphoketolase pathway employed to ferment carbohydrates. Much metabolic research on Bifidobacteria has focused on oligosaccharide metabolism as these carbohydrates are available in their otherwise nutrient-limited habitats.

Studies have shown that B. lactis alone or in combination with other compounds has a beneficial effect on gastrointestinal and immune functions. Specifically, it relieves constipation, improves fecal properties and microbiota, it has a positive effect against acute diarrhea and restores the intestinal microbiota after antibiotic treatments. Studies have further shown an advantage in treating colic and reducing the risk of rotavirus diarrhea in infants.

In various embodiments, a probiotic relates to microorganisms (e.g., bacteria, yeast, fungus and/or virus) which may form a portion of host flora, for example transient flora, and/or which may confer a therapeutic benefit to a host, for example when administered in adequate amounts. Non-limiting examples of probiotic bacteria include Lactobacillus plantarum, Lactobacillus acidophilus, Lactobacillus paracasei, Leuconostoc mesenteroides, Lactobacillus bulgaricus, Lactobacillus casei, Lactobacillus salivarius, Pediococcus pentosaceus, Streptococcus thermophiles, Bacillus subtilis, Bacillus coagulans, and Enteroccous faecium.

In some embodiments, the probiotic bacteria is B. lactis. B. lactis, as described above, refers to a particular strain of Bifidobacterium probiotic bacteria. In various embodiments, the prebiotic (e.g., a fungal extract such as AHCC or K1) and B. lactis composition may be administered with additional probiotic bacteria. In some embodiments, the probiotic bacteria is a Bifidobacterium selected from the group consisting of Bifidobacterium adolescentis, Bifidobacterium animalis, Bifidobacterium asteroids, Bifidobacterium bifidum, Bifidobacterium breve, Bifidobacterium catenulatum, Bifidobacterium infantis, Bifidobacterium longum and Bifidobacterium pseudocatenulatum. In some embodiments, the probiotic comprises one or more strains of one or more species of Bifidobacterium.

In some embodiments, the probiotic bacteria are species of the genus Lactobacillus. In some embodiments, the probiotic bacteria are selected from the group consisting of Lactobacillus rhamnosus and Lactobacillus reuteri. In some embodiments, the probiotic comprises one or more strains of one or more species of Lactobacillus.

In some embodiments, the probiotic comprises one or more species and/or strains of Bifidobacterium and/or Lactobacillus either alone or in combination. For example, in some embodiments, the probiotic comprises a mixture of Lactobacillus rhamnosus and Lactobacillus reuteri. In some embodiments, the probiotic comprises a mixture of Lactobacillus rhamnosus strain GR-1 and Lactobacillus reuteri strain RC-14.

The probiotic bacteria of the present disclosure may also include mutant, variant, and genetically modified mutants of probiotic bacteria strains whose genetic and/or phenotypic properties are altered compared to the parent strain. Naturally occurring variants of probiotic bacteria strains include the spontaneous alterations of targeted properties selectively isolated while deliberate alteration of parent strain properties is accomplished by conventional genetic manipulation technologies, such as gene disruption, conjugative transfer, etc.

The general state of probiotic bacteria is in the form of viable cells, or freeze-dried cells (which was used to generate the data herein). However, it can also be extended to non-viable cells such as killed cultures or compositions containing beneficial factors expressed by the probiotic bacteria. This could include thermally killed micro-organisms or micro-organisms killed by exposure to altered pH or subjection to pressure. With non-viable cells product preparation is simpler, cells may be incorporated easily into pharmaceuticals and storage requirements are much less limited than viable cells.

### Compositions and Pharmaceutical Formulations

The present disclosure relates to the unexpected discovery that certain compositions comprising prebiotics and probiotic bacteria are beneficial to a subject's immune system. In some embodiments, compositions comprising prebiotics and probiotic bacteria improve a subject's immune system function. In some embodiments, compositions comprising prebiotics and probiotic bacteria enhance a subject's immune system function. In some embodiments, compositions comprising prebiotics and probiotic bacteria restore a subject's dysfunctional immune system. In some embodiments, compositions comprising prebiotics and probiotic bacteria control a subject's overactive immune system. In some embodiments, compositions comprising prebiotics and probiotic bacteria maintain a subject's immunity over a period of time.

In some embodiments, the compositions of the present disclosure comprise a combination of a prebiotic and a probiotic bacteria. In some embodiments, the compositions comprise AHCC as the prebiotic and B. lactis as the probiotic. In some embodiments, the compositions comprise K1 as the prebiotic and B. lactis as the probiotic. In some embodiments, the compositions comprise both AHCC and K1 as the prebiotic and B. lactis as the probiotic. In some embodiments, the compositions comprise one or both of AHCC and K1 as the prebiotic and one or more of any probiotics disclosed herein, or generally known. In some embodiments, the probiotic ingredient in the combination may be an extract of a probiotic microbe, such as an isolated cell wall fraction or an isolated metabolite produced by a probiotic culture.

In some embodiments, the prebiotic promotes growth of bacteria in the gut, increase adhesion of probiotic bacteria in the gut, displace pathogens, or provide a fermentable dose of carbohydrate to probiotic bacteria (symbiotic) or selected commensal bacteria and increase the levels of those microbial populations in the gastrointestinal tract. Non-limiting examples of microbial populations include lactobacilli and bifidobacteria.

In some embodiments, the amount of prebiotic in the composition ranges from about 1 mg to about 0.5 g. In some embodiments, the amount of prebiotic in the composition ranges from about 0.5 g to about 5 g. In some embodiments, the amount of prebiotic in the composition ranges from about 1 g to about 3 g. In some embodiments, the amount of prebiotic in the composition is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, or 10 g, or a value within a range defined by any two of the aforementioned values. In some embodiments, a therapeutically effective amount of prebiotic is about 0.1 mg to about 5 g. In some embodiments, the therapeutically effective amount of is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, or 10 g, or a value within a range defined by any two of the aforementioned values.

In some embodiments, the dose of prebiotic ranges from about 1 mg/day to about 0.5 g/day. In some embodiments, the dose of prebiotic ranges from about 0.5 g/day to about 5 g/day. In some embodiments, the dose of prebiotic ranges from about 1 g/day to about 3 g/day. In some embodiments, the dose of prebiotic ranges from about 1 mg/day to about 0.5 mg/day. In some embodiments, e amount of prebiotic in the composition is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, or 10 mg/day, or a value within a range defined by any two of the aforementioned values.

In some embodiments, the dose of prebiotic ranges from about 0.1 mg/kg to about 100 mg/kg based on an average human weight of about 60 kg. In some embodiments, the dose of prebiotic ranges from about 0.5 mg/kg to about 50 mg/kg based on an average human weight of about 60 kg. In some embodiments, the dose of prebiotic ranges from about 25 mg/kg to about 150 mg/kg based on an average human weight of about 60 kg. In some embodiments, the dose of prebiotic is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg/kg, or a value within a range defined by any two of the aforementioned values.

In some embodiments, the amount of AHCC in the composition ranges from about 0.5 mg to about 5 g. In some embodiments, the amount of AHCC in the composition is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, or 10 g, or a value within a range defined by any two of the aforementioned values. In some embodiments, the dose of AHCC ranges from about 0.5 mg/day to about 5 g/day. In some embodiments, the dose of AHCC is about 1800 mg/day. In some embodiments, the dose of AHCC is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, 10 50, 100, 250, 500, 750, 1000, 1500, 2000, 2500, 3000, 3500, 4000, 4500, or 5000 mg/day, or a value within a range defined by any two of the aforementioned values. In some embodiments, the dose of AHCC ranges from about 0.1 mg/kg to about 150 mg/kg based on an average human weight of about 60 kg. In some embodiments, the dose of AHCC is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg/kg, or a value within a range defined by any two of the aforementioned values.

In some embodiments, the amount of K1 in the composition ranges from about 0.5 mg to about 5 g. In some embodiments, the amount of K1 in the composition is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, or 10 g, or a value within a range defined by any two of the aforementioned values. In some embodiments, the dose of K1 ranges from about 0.5 mg/day to about 5 g/day. In some embodiments, the dose of K1 is about 1800 mg/day. In some embodiments, the dose of K1 is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, 10 50, 100, 250, 500, 750, 1000, 1500, 2000, 2500, 3000, 3500, 4000, 4500, or 5000 mg/day, or a value within a range defined by any two of the aforementioned values. In some embodiments, the dose of K1 ranges from about 0.1 mg/kg to about 150 mg/kg based on an average human weight of about 60 kg. In some embodiments, the dose of K1 is about 0.01, 0.025, 0.05, 0.075, 0.1, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg/kg, or a value within a range defined by any two of the aforementioned values.

In some embodiments, the amount of probiotic in the composition ranges from about 0.5 million CFU to about 100 billion CFU. In some embodiments, the amount of probiotic in the composition ranges from about 1 billion CFU to about 50 billion CFU. In some embodiments, the amount of probiotic in the composition ranges from about 50 billion CFU to about 100 billion CFU. In some embodiments, the amount of probiotic in the composition is about 0.001, 0.0025, 0.005, 0.0075, 0.01, 0.025, 0.05, 0.075, 0.1, 0.25, 0.5, 0.75, 1, 10, 25, 50, 75, 100, 250, 500, 750 million, 1 billion, 10 billion, 25 billion, 50 billion, 75 billion, 100 billion, 250 billion, or 500 billion CFU, or a value within a range defined by any two of the aforementioned values.

In some embodiments, the dose of probiotic ranges from about 0.5 million CFU/day to about 100 billion CFU/day. In some embodiments, the dose of probiotic ranges from about 0.5 million CFU/day to about 1 billion CFU/day. In some embodiments, the dose of probiotic ranges from about 1 billion CFU/day to about 50 billion CFU/day. In some embodiments, the dose of probiotic ranges from about 50 billion CFU/day to about 100 billion CFU/day. In some embodiments, the dose of probiotic is about 0.001, 0.0025, 0.005, 0.0075, 0.01, 0.025, 0.05, 0.075, 0.1, 0.25, 0.5, 0.75, 1, 10, 25, 50, 75, 100, 250, 500, 750 million, 1 billion, 10 billion, 25 billion, 50 billion, 75 billion, 100 billion, 250 billion, or 500 billion CFU/day, or a value within a range defined by any two of the aforementioned values.

In some embodiments, the dose of probiotic ranges from about 10,000 CFU/kg to about 10 million CFU/kg. In some embodiments, the dose of probiotic ranges from about 100,000 CFU/kg to about 1 million CFU/kg based on an average human weight of about 60 kg. In some embodiments, the dose of probiotic ranges from about 1 million CFU/kg to about 10 million CFU/kg based on an average human weight of about 60 kg. In some embodiments, a therapeutically effective amount of probiotic is about 0.5 million CFU to about 100 billion CFU based on an average human weight of about 60 kg. In some embodiments, the dose of probiotic is about 500, 1,000, 2,500, 5,000, 7,500, 10,000, 25,000, 50,000, 100,000, 250,000, 500,000, 750,000, 1 million, 2.5, million, 5 million, 7.5 million, 10 million, 25 million, 50 million, 75 million, 100 million, 250 million, 500 million, 750 million, 1 billion, or 1.25 billion CFU/kg, or a value within a range defined by any two of the aforementioned values.

In some embodiments, the amount of B. lactis in the composition ranges from about 0.5 million CFU to about 10 billion CFU. In some embodiments, the dose of B. lactis ranges from about 0.5 million CFU/day to about 10 billion CFU/day. In some embodiments, the dose of B. lactis is about 12 billion CFU/day. In some embodiments, the dose of AHCC ranges from about 10,000 CFU/kg to about 10 million CFU/kg based on an average human weight of about 60 kg. Various proportions of prebiotic and probiotic are contemplated in the compositions according to the present disclosure. For example, in some embodiments, compositions can comprise from about 0.5% to about 99.5% prebiotic and about 0.5% to about 99.5% probiotic. The proportions of prebiotic and probiotic in the compositions can be adjusted based on need by one of ordinary skill in the art. For example, in some embodiments, compositions can comprise about 5% prebiotic and about 95% probiotic. In some embodiments, compositions can comprise about 10% prebiotic and about 90% probiotic. In some embodiments, compositions can comprise about 15% prebiotic and about 85% probiotic. In some embodiments, compositions can comprise about 20% prebiotic and about 80% probiotic. In some embodiments, compositions can comprise about 25% prebiotic and about 75% probiotic. In some embodiments, compositions can comprise about 30% prebiotic and about 70% probiotic. In some embodiments, compositions can comprise about 35% prebiotic and about 65% probiotic. In some embodiments, compositions can comprise about 40% prebiotic and about 55% probiotic. In some embodiments, compositions can comprise about 50% prebiotic and about 50% probiotic. In some embodiments, compositions can comprise about 55% prebiotic and about 45% probiotic. In some embodiments, compositions can comprise about 60% prebiotic and about 40% probiotic. In some embodiments, compositions can comprise about 65% prebiotic and about 35% probiotic. In some embodiments, compositions can comprise about 70% prebiotic and about 30% probiotic. In some embodiments, compositions can comprise about 75% prebiotic and about 25% probiotic. In some embodiments, compositions can comprise about 80% prebiotic and about 20% probiotic. In some embodiments, compositions can comprise about 85% prebiotic and about 15% probiotic. In some embodiments, compositions can comprise about 90% prebiotic and about 10% probiotic. In some embodiments, compositions can comprise about 95% prebiotic and about 5% probiotic.

In some embodiments, pharmaceutical formulations comprising one or more compositions are disclosed. The pharmaceutical formulations described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit formulations may comprise additional medicinal agents, pharmaceutical agents, carriers, buffers, adjuvants, dispersing agents, diluents, and the like depending on the intended use and application.

Examples of suitable pharmaceutical carriers, excipients and/or diluents are well known in the art and include, but are not limited to, a gum, a starch (e g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulations are aqueous or non-aqueous solutions, suspensions, emulsions or oils, Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, turmeric oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Formulations comprising such carriers can be formulated by well-known conventional methods. Suitable carriers. may comprise any material which, when combined with the biologically active compounds of the disclosure, retains the biological activity. Preparations for parenteral administration may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like, in addition, the pharmaceutical formulations of the present disclosure might comprise proteinaceous carriers, e.g., serum albumin or immunoglobulin, preferably of human origin.

The pharmaceutical formulations provided herein may also be administered as controlled-release formulations, i.e. formulations in which the active ingredient is released over a period of time after administration. Controlled- or sustained-release formulations include formulation in lipophilic depots (e.g. fatty acids, waxes, oils). In another embodiment, the formulation is an immediate-release formulation, i.e. a formulation in which all the active ingredient is released immediately after administration.

Further, the pharmaceutical formulations may be administered admixed to food, functional food, drinks, medicinal food.

Although the description of pharmaceutical formulations provided herein are principally directed to pharmaceutical formulations which are suitable for ethical administration to humans, it will be understood by the skilled artisan that such formulations are generally suitable for administration to animals of all sorts. Modification of pharmaceutical formulations suitable for administration to humans in order to render the formulations suitable for administration to various animals is well understood, and the ordinarily skilled veterinary pharmacologist can design and perform such modification with merely ordinary, if any, experimentation. Subjects to which administration of the pharmaceutical formulations of the disclosure is contemplated include, but are not limited to, humans and other primates, mammals including commercially relevant mammals such as non-human primates, cattle, pigs, horses, sheep, cats, dogs, duck, chicken, and sheep.

Pharmaceutical formulations of the disclosure may be prepared, packaged, or sold in bulk, as a single unit dose, or as a plurality of single unit doses. As used herein, a “unit dose” is discrete amount of the pharmaceutical formulations comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of an active ingredient, a pharmaceutically acceptable carrier, and any additional ingredients in a pharmaceutical formulation of the disclosure will vary, depending upon the identity, size, and condition of the subject treated and further depending upon the route by which the formulation is to be administered. By way of example, the formulation may comprise between 0.1% and 100% (w/w) active ingredient.

In addition to the active ingredients, a pharmaceutical formulation of the disclosure may further comprise one or more additional pharmaceutically active agents.

Controlled- or sustained-release pharmaceutical formulations may be made using conventional technology.

Formulations of the present disclosure may also comprise a prebiotic component. “Prebiotic” includes substances or compounds that are fermented by the intestinal flora of the pet and hence promote the growth or development of lactic acid bacteria in the gastro-intestinal tract of the pet at the expense of pathogenic bacteria. The result of this fermentation can be a release of fatty acids, in particular short-chain fatty acids in the colon. This release can have the effect of reducing the pH value in the colon. Non-limiting examples of suitable prebiotics include oligosaccharides, such as inulin and its hydrolysis products commonly known as fructooligosaccharides, galacto-oligosaccarides, xylo-oligosaccharides, or oligo derivatives of starch (such as pectin, beta-glucan, and resistant starch). The prebiotics may be provided in any suitable form.

For example, the prebiotic may be provided in the form of plant material that contains the fiber. Suitable plant materials include asparagus, artichokes, onions, wheat or chicory, or residues of these plant materials. Alternatively, the prebiotic fiber may be provided as an inulin extract, for example extracts from chicory are suitable. Suitable inulin extracts may be obtained from Orafti SA of Tirlemont 3300, Belgium under the trade mark “Raftiline”. For example, the inulin may be provided in the form of Raftiline (g) ST which is a fine white powder, which contains about 90 to about 94% by weight of inulin, up to about 4% by weight of glucose and fructose, and about 4 to 9% by weight of sucrose. Alternatively, the fiber may be in the form of a fructooligosaccharide such as obtained from Orafti SA of Tirlemont 3300, Belgium under the trade mark “Raftilose”. For example, the inulin may be provided in the form of Raftilose (g) P95. Otherwise, the fructooligosaccharides may be obtained by hydrolyzing inulin, by enzymatic methods, or by using micro-organisms.

Pharmaceutical formulations also include nutritional formulations, such as oral nutritional formulations for oral consumption and optionally for enteral adsorption, wherein the nutritional formulation includes the compounds of the present disclosure.

If the nutritional formulations are formulated to be administered orally, the formulations may be a liquid oral nutritional formulation. In some embodiments, the oral nutritional formulation in an incomplete nutritional formulation. In some embodiments, the oral nutritional formulation is a complete nutritional formulation. In this manner, the nutritional formulations may be administered in any known form including, for example, tablets, capsules, liquids, chewables, soft gels, sachets, powders, syrups, liquid suspensions, emulsions and solutions in convenient dosage forms.

A nutritional formula encompasses any nutritionally complete or nutritionally incomplete (for e.g., a supplementary formulation, a nutritional supplement). As used herein, “nutritionally complete” are preferably nutritional products that contain sufficient types and levels of macronutrients (protein, fats and carbohydrates) and micronutrients to be sufficient to be a sole source of nutrition for the subject to which it is being administered to. Patients can receive 100% of their nutritional requirements from such complete nutritional formulations. According to some embodiments, the nutritional formula is a supplementary formulation providing supplementary nutrition. A “supplementary formula” may not be nutritionally complete, but preferably contains specific nutrients that are supportive, for example in combination with physical exercise, which further the beneficial effects of the disclosure, and/or which address specific or additional needs of the subject.

The nutritional formula may be a generally applicable nutritional formula, for example adapted to subjects of a specific age, for example a formula for children, but it may also be a formula for elderly patients, for intensive care patients, or a specially adapted formula for patients suffering from a specific disease, for example. Any nutritional formula may be reconstitutable, that is, present in a substantially dried, for example powdered form, or ready-to-drink, in the form of liquid formulas, for example.

### Kits

The disclosure also includes one or more kits comprising any of the compositions or pharmaceutical formulations useful within the methods of the disclosure and an instructional material that describes, for instance, the method of administering AHCC or K1, and B. lactis as described elsewhere herein, or the method of administering prebiotics and probiotics as described elsewhere herein. Pharmaceutical formulations suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen free water) prior to parenteral administration of the reconstituted formulations.

The pharmaceutical formulations may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer system. Formulations for sustained release or implantation may comprise pharmaceutically acceptable polymeric or hydrophobic materials such as an emulsion, an ion exchange resin, a sparingly soluble polymer, or a sparingly soluble salt.

In some embodiments, pharmaceutical compositions comprising prebiotics and probiotics of the present disclosure in combination with one or more other therapeutic agents such as an anti-tumor agent, a chemotherapeutic agent, an anti-cell proliferation agent, an anti-tumor vaccine and the like are also contemplated. For example, in some embodiments, pharmaceutical compositions comprising AHCC and B. lactis in combination with one or more other therapeutic agents such as an anti-tumor agent, a chemotherapeutic agent, an anti-cell proliferation agent, an anti-tumor vaccine and the like are also contemplated. Likewise, in some embodiments, pharmaceutical compositions comprising K1 and B. lactis in combination with one or more other therapeutic agents such as an anti-tumor agent, a chemotherapeutic agent, an anti-cell proliferation agent, an anti-tumor vaccine and the like are also contemplated.

### Methods of Treatment

In various embodiments, the present disclosure provides methods of treatment using the compositions of the disclosure. In some embodiments, the methods relate to methods of enhancing the immune system. In some embodiments, the methods relate to methods of enhancing the immune response to vaccination. In some embodiments, the method relates to enhancing the immune response to treat a disease or disorder that is associated with dysfunctional immune response. In some embodiments, the methods relate to methods of enhancing the immune response to treat cancer. In some embodiments, the methods relate to methods of enhancing the immune response to instances where the immune system is challenged or compromised, such as in therapy, surgery and the likes.

In some embodiments, the disclosure relates to generically treating diseases or disorders associated with dysfunctional immune response whereby having one or more beneficial effect on the immune system is a desired therapeutic outcome. Non-limiting examples of beneficial effects on a subject's immune system include increase in red blood cell count, increase in hemoglobin content, increase in hematocrit, improved antigen presentation and improved immunoglobulin secretion. Other non-limiting examples of beneficial effects on a subject's immune system macrophage antigen presentation activity and inhibition of tumor-derived immune suppressive factors, enhance macrophage proliferation and activation, promote differentiation of Th1 cells; increase macrophage production of IL-12, increase NK activity; promote apoptosis of cancer cells. AHCC in cancer patients has been reported to increase TNF-a, y-interferon, interleukin-12 and decrease immunosuppressive acidic protein (IAP) and tumor growth factor (TGF)-a.

In some embodiments, the methods relate to the administration of a therapeutic amount of the compositions of the disclosure. For example, in certain instances, a subject may be administered from 0.5 mg to 100 g of the compositions of the disclosure. Preferably, subject may be administered from 1 mg to 12 g of the compositions of the disclosure.

In some embodiments, the combination of prebiotic and probiotic produces an additive beneficial effect on the immune system, such that the prebiotic and probiotic ingredients in the combination behave as they are observed to behave when given by themselves—where neither ingredient modifies the effect of the other. In some embodiments, the combination of prebiotic and probiotic produces an unexpected and surprising synergistic effect. Such synergistic effects are greater than additive effects. An additive effect is observed when the effect on the immune system is equal to the sum of the individual effects of the prebiotic component and the probiotic component. A synergistic effect is observed when the potentiation is greater than the sum of the individual effects of the prebiotic component and the probiotic component. Synergistic effect, additive effect or both can occur in human patients, non-human patients, non-patient human volunteers, in vivo models, ex vivo models, in vitro models, etc. Potentiation can range from about <1 to about 100-fold. In some embodiments, the synergistic effect is about 2 to about 75-fold. In some embodiments, the potentiation ranges from <1, 1, >1, 2, 3, 4, 5, 6, 7, 8, 9, 10, 20, 30, 40, 50, 60, 70, 80, 90, or 100-fold, or within a range defined by any two of the aforementioned values. A synergistic effect allows one or all of the components of a composition to be used in amounts and/or doses that are lower that the amounts and/or doses of doses of the components used individually. Thus, in some embodiments, the prebiotic can potentiate the effect of the probiotic on the immune system. In some embodiments, the probiotic can potentiate the effect of the prebiotic on the immune system. In some embodiments, the prebiotic can potentiate the effect of the probiotic and the probiotic can potentiate the effect of the prebiotic on the immune system. The potentiation can occur in several ways. Non-limiting examples include enhancing the effectiveness of an already effective prebiotic and/or probiotic, making an ineffective prebiotic and/or probiotic effective (the prebiotic and/or probiotic could also have been previously effective but become ineffective following long term and/or short term use in a patient), increasing the length of time for which a prebiotic and/or probiotic is effective, decreasing the effective dose of administration of a prebiotic and/or probiotic, decreasing the duration of time for which a prebiotic and/or probiotic is administered, decreasing the frequency of administration of a prebiotic and/or probiotic, and/or enabling the administration of a prebiotic and/or probiotic via one or more amenable route.

### Immune Activation/Methods of Enhancing Immune System

Without being limited by any particular theory, an initial step was the identification of a unique and novel medicinal mushroom-based extract designated K1 (disclosed herein and in U.S. Provisional Application No. 62/663,878, entitled “Bioactive Fungal Compounds Produced By Solid-State Fermentation,” filed on Apr. 27, 2018, and incorporated herein in its entirety by reference thereto). Without being limited by any particular theory, it was previously shown that mushroom extract, such as AHCC, interacted synergistically with probiotic bacteria (as disclosed in the co-pending U.S. Provisional Application No. 62/505,641, entitled “Nutraceutical Blends,” filed on May 12, 2017, and incorporated herein in its entirety by reference thereto). Therefore, a subsequent strategy was to evaluate whether the biological and immune-modulating properties of K1 might be affected and/or improved by co-administration with probiotics (e.g., probiotic bacteria).

Without being limited by any particular theory, the rationale for combining K1 with a probiotic (e.g., probiotic bacteria) was to potentially activate a synergistic immune activation cascade, wherein mushroom-derived alpha-glucans and beta-1,3 and -1,6 branched glucans would engage immune cell surface receptors simultaneous to linear bacterial beta-1,3 glucans. Immune cells, including macrophages, lymphocytes, and dendritic cells, have cell surface receptors called pattern recognition receptors. Glucans are not able to enter cells, but rather mediate their potent immune activating functions by binding to multiple types of such cell surface receptors. Additional benefits of consuming probiotic bacteria include, without limitations, their secreted metabolites engaging in cross-talk with the gut mucosal immune cells. Such probiotic secreted metabolites have demonstrated both immune activating and also anti-inflammatory effects in cell-based bioassays.

Without being limited by any particular theory, when comparing K1 alone with a combination of K1 and probiotic fractions, the combination showed superior biological and immune-modulating properties compared to each ingredient alone. The synergistic effects seen when K1 was blended with probiotic fractions included, without limitations, synergistic activation of multiple types of immune cells, synergistic production of immune-activating, anti-inflammatory, and regenerative biomarkers, synergistic reduction in breast cancer cell viability.

Thus, in some embodiments, methods of prevention and/or treatment of one or more diseases by providing combinations of prebiotic and probiotic compounds, and the compositions and/or kits thereof, to a subject in need thereof are disclosed. In some embodiments, methods of enhancing a subject's immune system by providing combinations of prebiotic and probiotic compounds, and the compositions and/or kits thereof, are disclosed. In some embodiments, methods of enhancing a subject's immune response to vaccination by providing combinations of prebiotic and probiotic compounds, and the compositions and/or kits thereof, are disclosed.

In some embodiments, the fungal extracts disclosed herein activate one or more immune cells and induce and/or enhance the production of one or more cytokines by the one or more immune cells. In some embodiments, the fungal extract AHCC activates one or more immune cells and induces and/or enhances the production of one or more cytokines by the one or more immune cells. In some embodiments, the fungal extract K1 activates one or more immune cells and induces and/or enhances the production of one or more cytokines by the one or more immune cells. In some embodiments, the one or more immune cells activated by the fungal extracts AHCC and K1 are the same. In some embodiments, the one or more immune cells activated by the fungal extracts AHCC and K1 are different. In some embodiments, the one or more immune cells activated by the fungal extracts AHCC and K1 are overlapping. In some embodiments, the activation of the one or more immune cells by AHCC may be superior to the activation by K1. In some embodiments, the activation of the one or more immune cells by K1 may be superior to the activation by AHCC.

In some embodiments, an immune activation by a combination of prebiotic and probiotic is additive. In some embodiments, an immune activation by a combination of prebiotic and probiotic is synergistic. In some embodiments, the probiotic is whole cells (e.g., whole bacteria), such as B. lactis. In some embodiments, the probiotic is one or more cellular fractions of the probiotic. Non-limiting examples, of one or more cellular fractions include cell wall preparation/cell wall fraction, cellular metabolites fraction, cytoplasmic fraction, or combinations thereof. In some embodiments, the immune modulatory effect of AHCC is compared to immune modulatory effect of K1. In some embodiments, one or more commercially available probiotic bacteria are cultured to obtain a secreted probiotic metabolites fraction and/or cell wall fraction for testing. In some embodiments, metabolites and cell walls are isolated by one or more techniques and/or methods known in the art. In some embodiments, after the metabolites were harvested, the probiotic cell wall fraction was isolated.

In some embodiments, immune activating properties of K1 and AHCC were tested individually and in combination with the probiotic metabolites and cell wall fractions. In some embodiments, an immune modulation by a combination of prebiotic AHCC and probiotic cell wall fraction is additive. In some embodiments, an immune modulation by a combination of prebiotic AHCC and probiotic cell wall fraction is synergistic. In some embodiments, an immune modulation by a combination of prebiotic K1 and probiotic cell wall fraction is additive. In some embodiments, an immune modulation by a combination of prebiotic K1 and probiotic cell wall fraction is synergistic. In some embodiments, an immune modulation by a combination of prebiotic AHCC and probiotic cellular metabolites fraction is additive. In some embodiments, an immune modulation by a combination of prebiotic AHCC and probiotic cellular metabolites fraction is synergistic. In some embodiments, an immune modulation by a combination of prebiotic K1 and probiotic cellular metabolites fraction is additive. In some embodiments, an immune modulation by a combination of prebiotic K1 and probiotic cellular metabolites fraction is synergistic. In some embodiments, the immune modulation includes immune activation, inhibition and/or regulation of an immune response. In some embodiments, the immune modulation results in a beneficial effect, for example, suppression of breast cancer cell viability, killing of cancer cells, etc.

In some embodiments, immune cell activation includes one or more of cytokine induction, upregulating of expression of activation markers on immune cells, and induction of anti-inflammatory cytokines. In some embodiments, the immune cell activation is associated with reduced cellular viability of cancer cells.

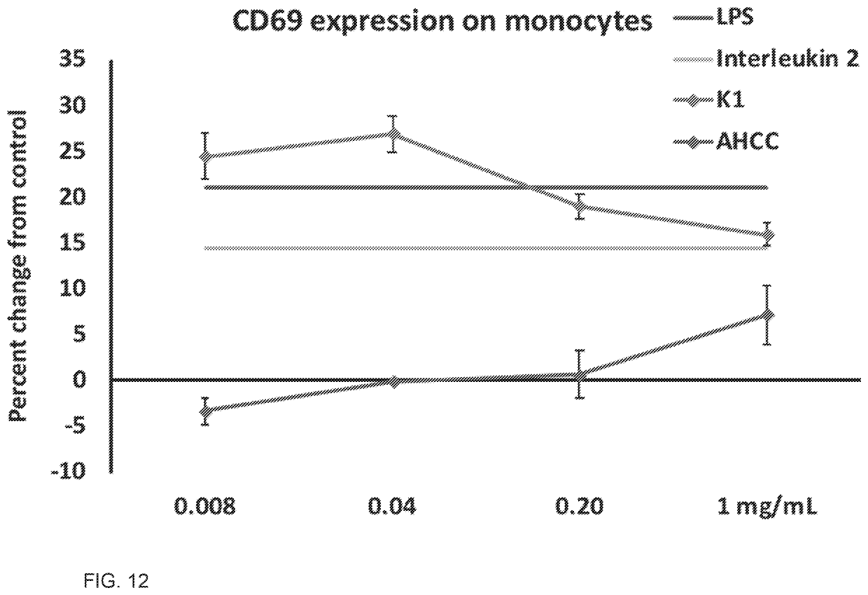

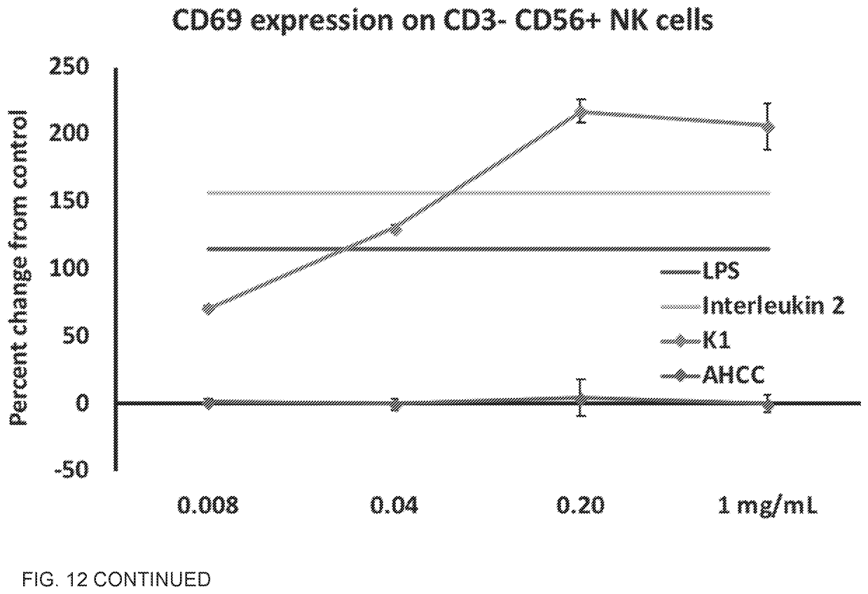

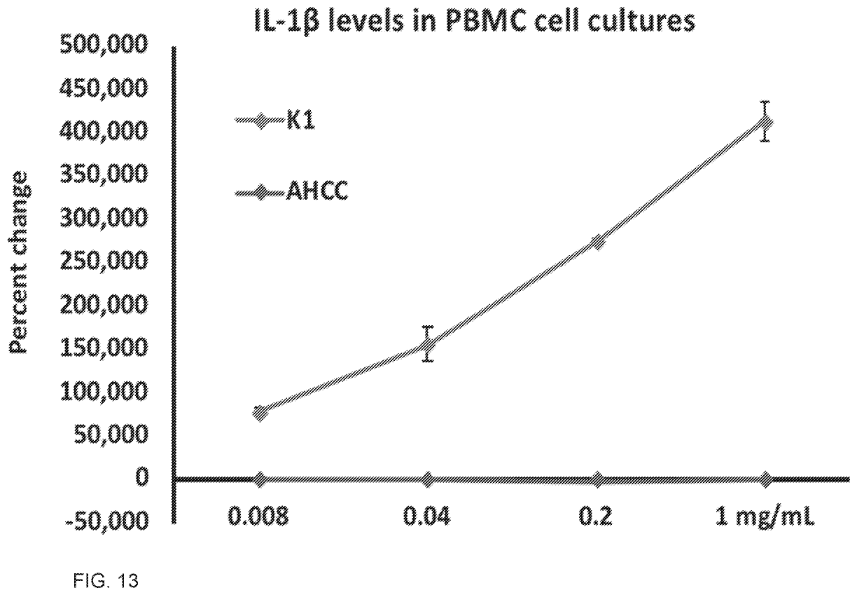

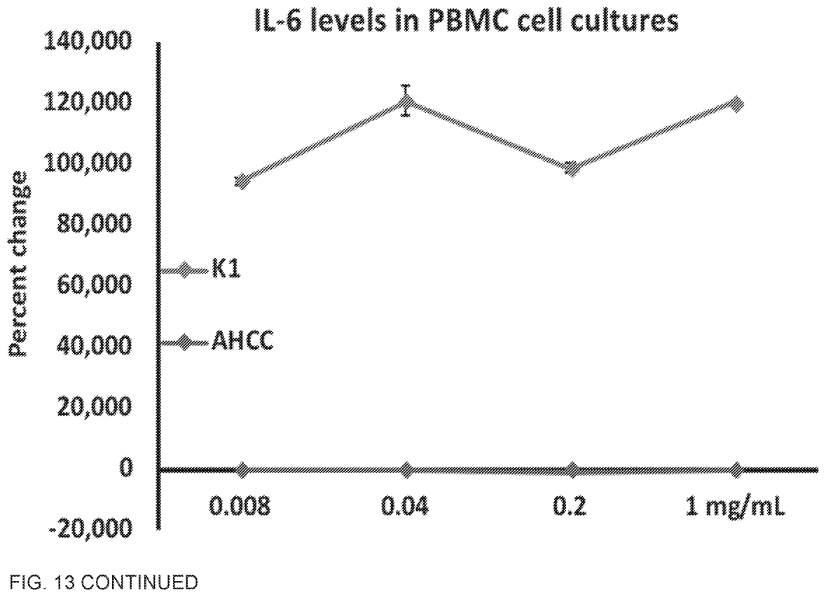

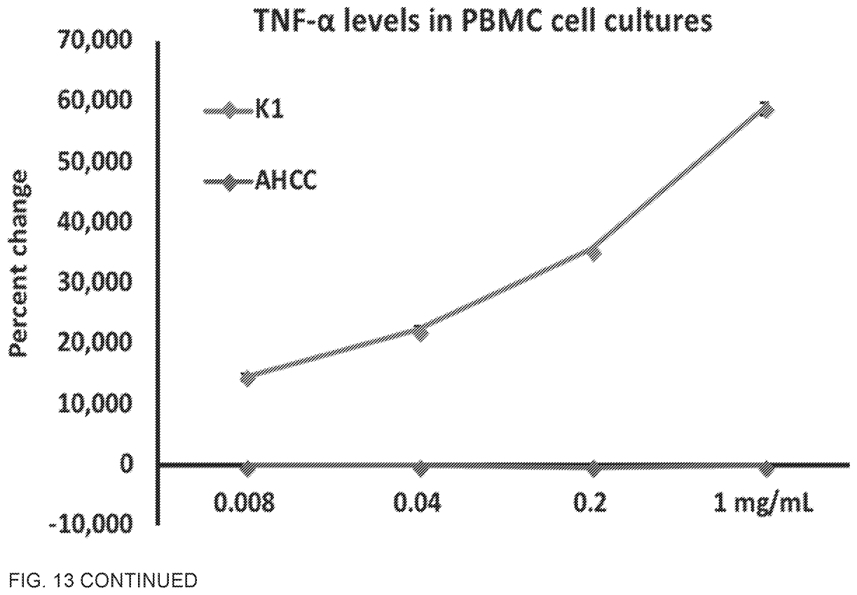

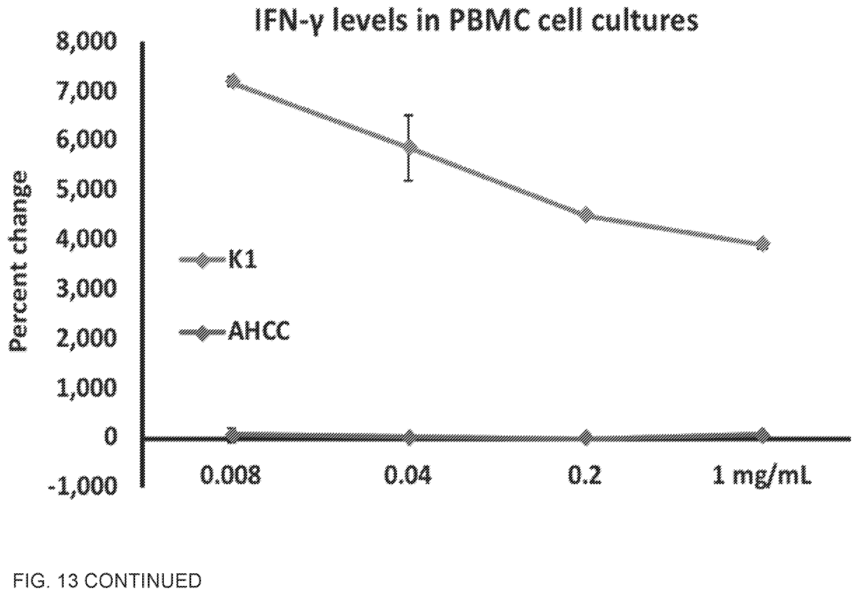

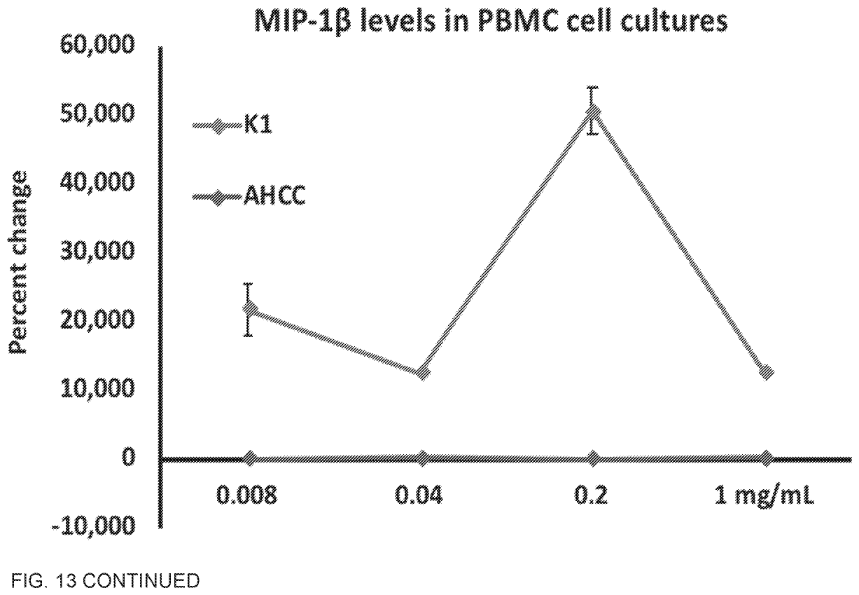

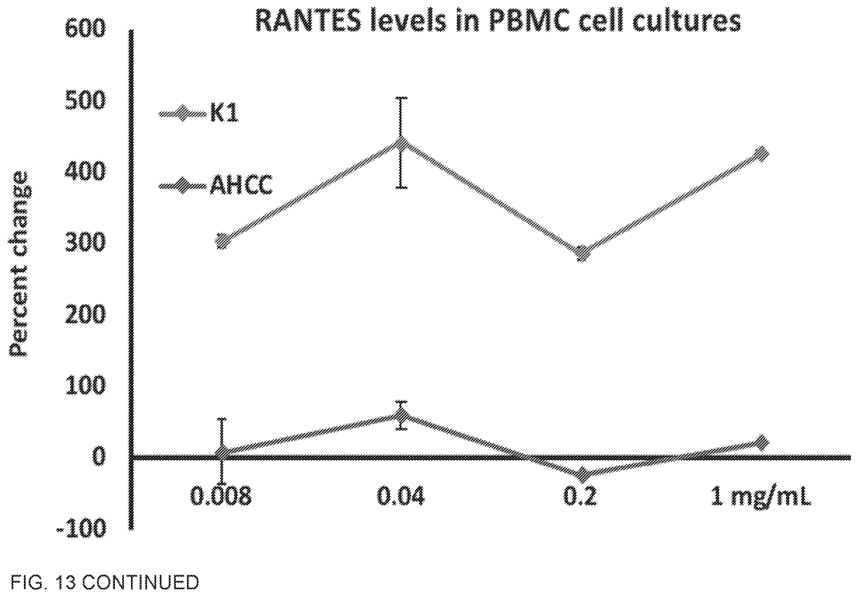

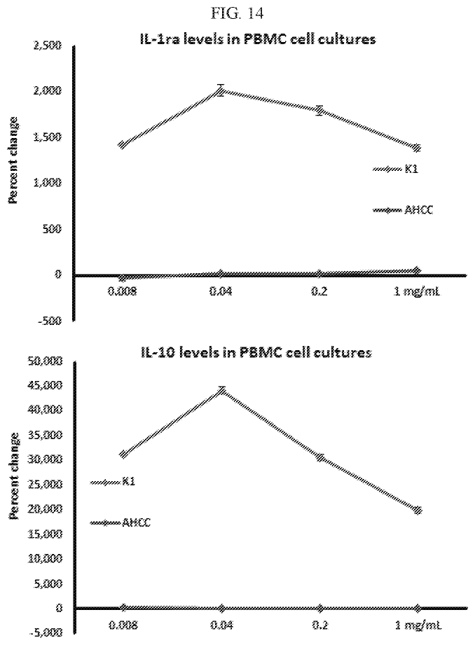

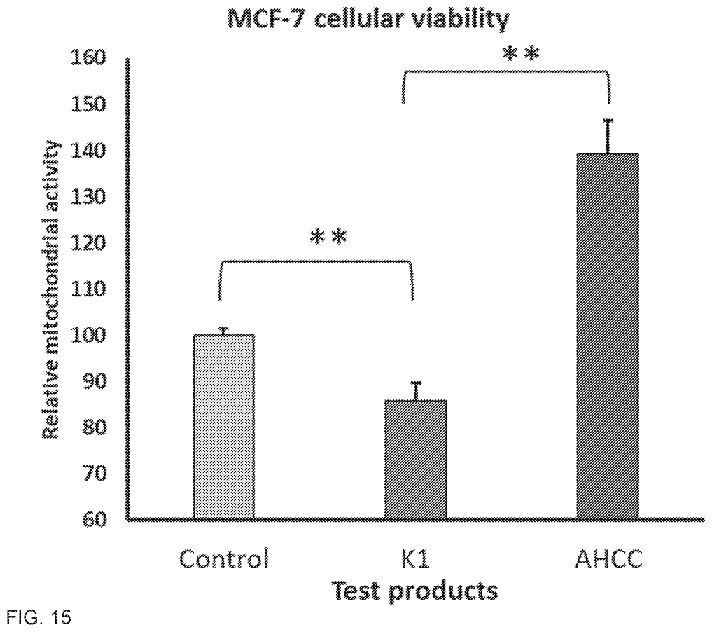

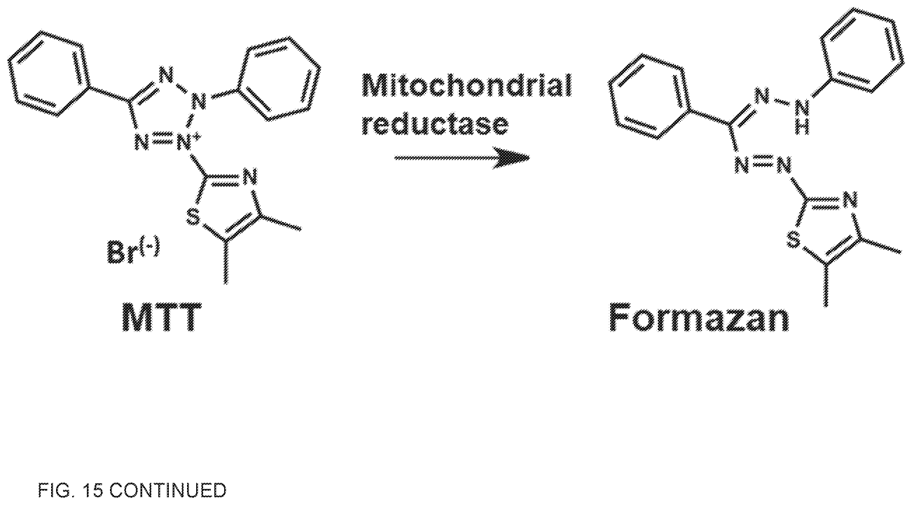

In some embodiments, K1 induces one or more activation markers on NK cells, monocytes and/or macrophages. For example, in some embodiments, K1 induces CD69 activation marker on NK cells, monocytes and/or macrophages (e.g., FIG. 12). In some embodiments, K1 upregulates the production of one or more immune-activating pro-inflammatory cytokines and/or chemokines (e.g., FIG. 13). For example, in some embodiments, K1 strongly induces the anti-inflammatory cytokine IL-1ra (e.g., FIG. 14). In some embodiments, K1 induces the anti-inflammatory cytokine IL-10 (e.g., FIG. 14). In some embodiments, K1 induces both IL-1ra and IL-10 (e.g., FIG. 14). In some embodiments, K1 directly reduces the cellular viability of a cancer cell line. For example, in some embodiments, K1 directly reduces the cellular viability of the breast cancer cell line MCF-7 (e.g., FIG. 15).

In some embodiments, K1 is better than AHCC in terms of immune cell activation and induction of cytokines. In some embodiments, AHCC is better than K1 in terms of immune cell activation and induction of cytokines. In some embodiments, both K1 and AHCC are on par in terms of immune cell activation and induction of cytokines.

In some embodiments, provided herein are methods for activating immune cells and/or inducing cytokines in a subject. In some embodiments, the subject is a human. In some embodiments, the subject is a non-human. In some embodiments, the method comprising administering to the subject an effective amount of a prebiotic. In some embodiments, the method comprising administering to the subject an effective amount of a probiotic. In some embodiments, the method comprising administering to the subject a combination of an effective amount of a prebiotic and an effective amount of one or more probiotics. In some embodiments, the prebiotic is the bioactive fungal extract AHCC. In some embodiments, the prebiotic is the bioactive fungal extract K1. In some embodiments, the combination comprises AHCC and one or more probiotics. In some embodiments, the combination comprises K1 and one or more probiotics. In some embodiments, the combination comprises AHCC, K1, and one or more probiotics.

In some embodiments, the probiotic is an extract, for example, an isolated cell wall fraction, an isolated metabolite produced by a probiotic, or a combination thereof.

In some embodiments, synergistic immune effects are obtained by a combination of a prebiotic (e.g., one or more novel mushroom extracts disclosed herein) and probiotic cell walls. In some embodiments, synergistic immune effects are obtained by a combination of a prebiotic (e.g., one or more novel mushroom extracts disclosed herein) and probiotic metabolites. In some embodiments, synergistic immune effects are obtained by a combination of a prebiotic (e.g., one or more novel mushroom extracts disclosed herein) and probiotic cell walls and metabolites.

In some embodiments, the present disclosure includes methods of enhancing the immune system by administering the compositions of the disclosure to a subject. The methods include methods of potentiating an immune response in normal subjects, in subjects who are immunocompromised, or in subjects who are at risk of infection due to disease, hospitalization, age or other predisposing medical factors. A method for activating immune cells and/or inducing cytokines in a subject in need thereof, the method comprising: administering to the subject an effective amount of a bioactive fungal extract, designated K1, wherein the effective amount is sufficient to produce superior immune cell activation and/or induction of cytokines compared to AHCC.

The methods of the present disclosure are effective in generally boosting the immune system in normal subjects. Normal subjects have a normally functioning immune system but may wish to enhance their immune system. For instance, normal subjects may use the methods of the present disclosure to maintain their health or as prophylaxis against possible immune system challenges.

In some embodiments, a subject's immune system is boosted by (i.e., a beneficial effect on a subject's immune system is by) an increase in red blood cell count, increase in hemoglobin content, increase in hematocrit, improved antigen presentation and improved immunoglobulin secretion.

In some embodiments, a beneficial effect on subject's immune system is observed in about 30 days to about 60 days. In some embodiments, a beneficial effect on subject's immune system is observed in about 10 days to about 30 days.

In some embodiments, a beneficial effect on subject's immune system lasts for about 60 days to about 360 days. In some embodiments, a beneficial effect on subject's immune system lasts for about 180 days to about 720 days. In some embodiments, a beneficial effect on subject's immune system lasts for more than about 720 days.

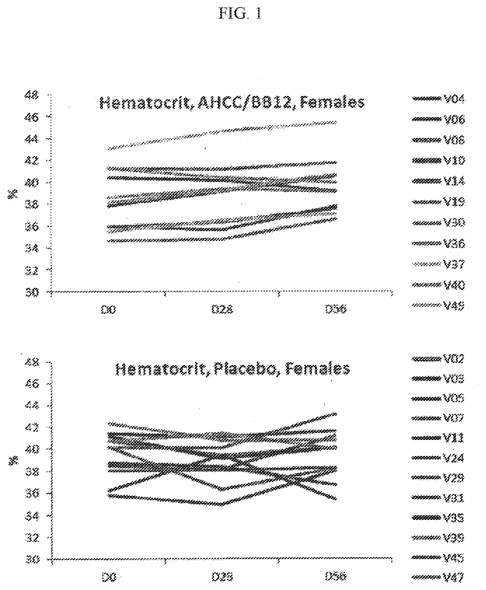

In some embodiments, the beneficial effect on a subject's immune system is in the form of an increase in hematocrit. FIG. 1 depicts the results of an experiment comparing the hematocrit results between female human subjects who ingested the composition of the present disclosure and female human subjects who ingested a placebo. The improvement from baseline in the group that ingested the composition of the present disclosure was statistically significant after only 4 weeks.

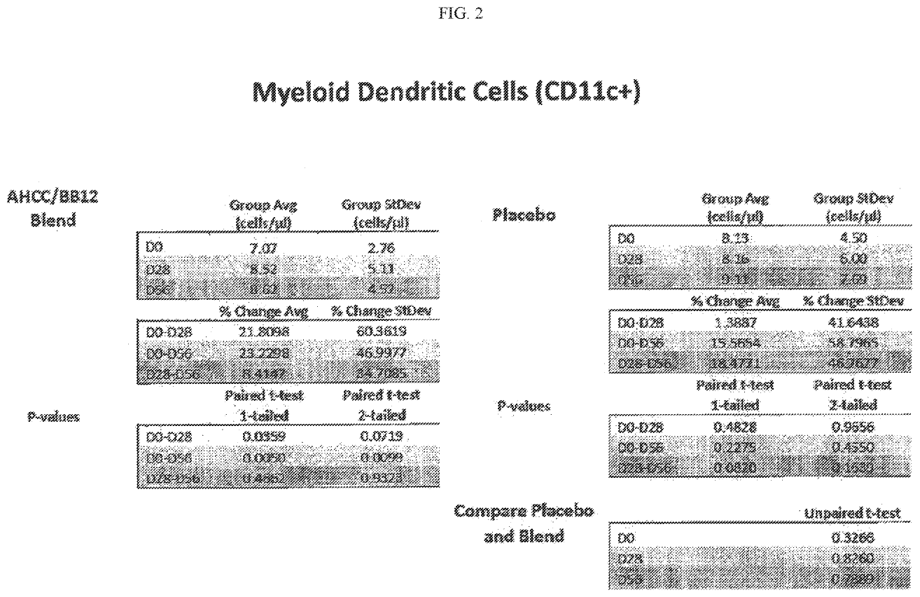

In some embodiments, the beneficial effect on a subject's immune system is in the form of an increase in CD11c+ myeloid dendritic cells. FIG. 2 depicts the results of an experiment comparing the number of CD11c+ myeloid dendritic cells between human subjects who ingested the composition of the present disclosure and human subjects who ingested a placebo. The group that ingested the composition of the present disclosure showed over 20% increase in myeloid dendritic cells at the end of 4 weeks, while no change was seen in the placebo group over the same period. This increase reached statistical significance in the group that ingested the composition at the end of 8 weeks. Thus, in some embodiments, the compositions disclosed herein cause an increase in CD11c+ myeloid dendritic cells by about 20%. In some embodiments, the increase in CD11c+ myeloid dendritic cells numbers ranges from about 5% to about 95%.

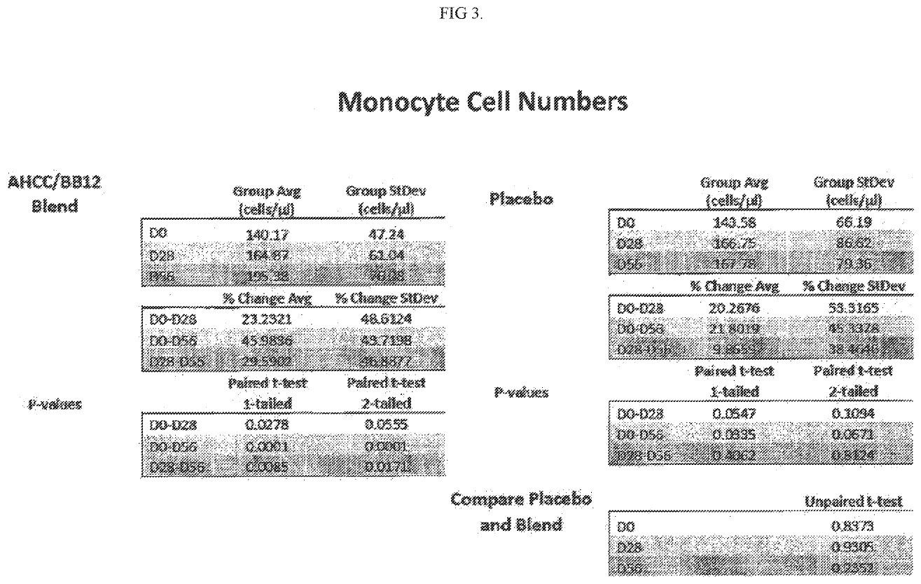

In some embodiments, the beneficial effect on a subject's immune system is in the form of an increase in monocytes. FIG. 3 depicts the results of an experiment comparing the number of monocyte cells between human subjects who ingested the composition of the present disclosure and human subjects who ingested placebo. At the end of 8 weeks, a statistically significant increase of 30% was seen in the group ingesting the composition, while only a minor, insignificant change was seen in the placebo group (p<0.0001). Thus, in some embodiments, the compositions disclosed herein cause an increase in monocytes by about 30%. In some embodiments, the increase in monocytes ranges from about 5% to about 95%.

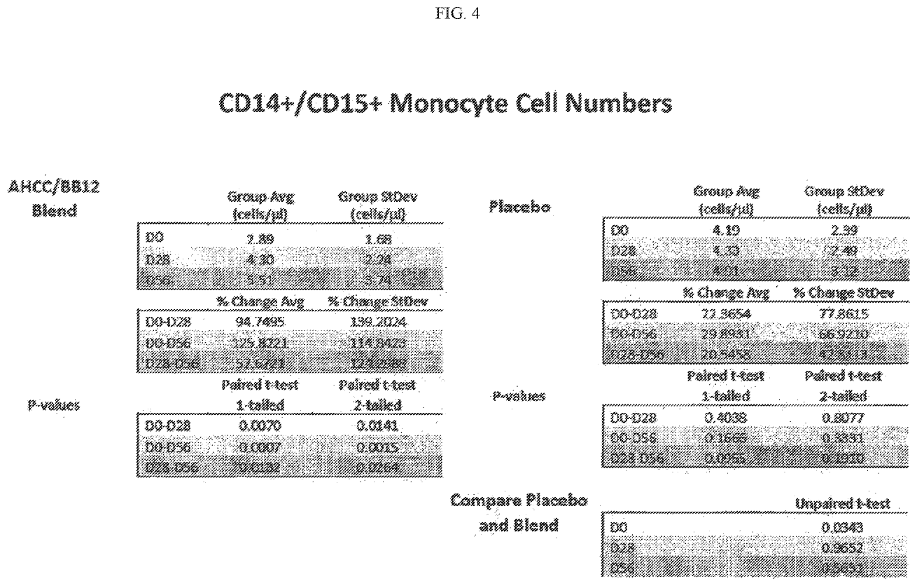

In some embodiments, the beneficial effect on a subject's immune system is in the form of an increase in CD14+CD16+ subset of monocyte cells. FIG. 4 depicts the results of an experiment comparing the number of CD14+CD16+ subset of monocyte cells between human subjects who ingested the composition of the present disclosure and human subjects who ingested placebo. A highly significant increase was seen in the group ingesting the composition for both the initial 4 week phase and for the phase following vaccine administration, while no change was seen in the placebo group. In some embodiments, the increase in CD14+CD16+ subset of monocyte cells ranges from about 5% to about 95%.

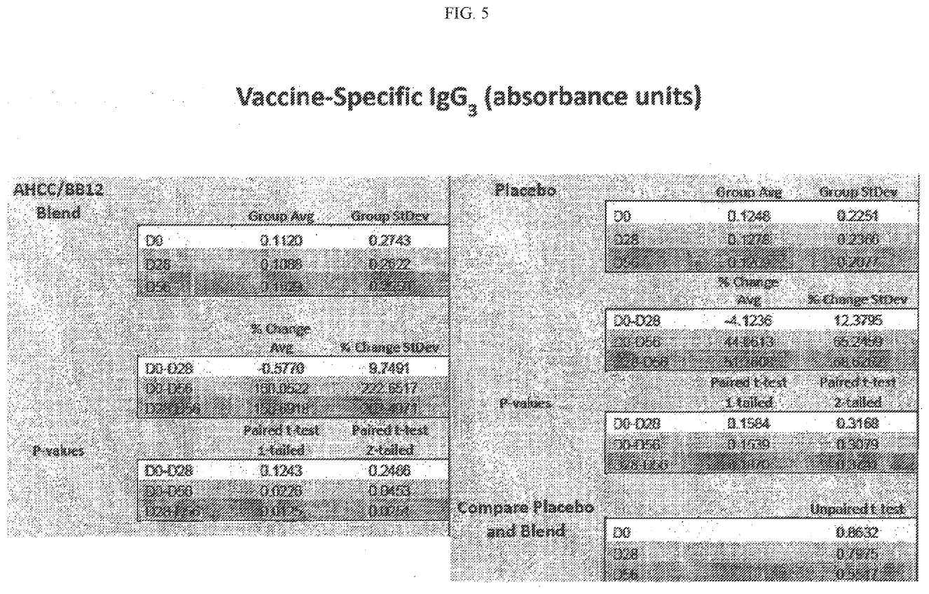

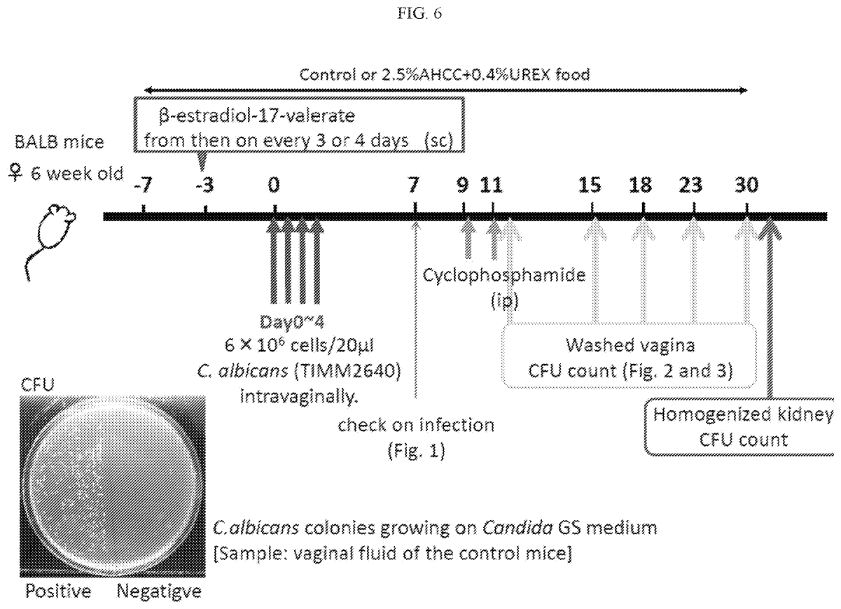

In some embodiments, the beneficial effect on a subject's immune system is in the form of an increase in levels of vaccine-specific IgG3. FIG. 5 depicts the results of an experiment comparing the level of vaccine-specific IgG3 between human subjects who ingested the composition of the present disclosure and human subjects who ingested placebo. After administering the vaccine, only the group ingesting the composition showed a statistically significant increase in the serum levels of vaccine-specific IgG3. In some embodiments, the increase in levels of vaccine-specific IgG3 ranges from about 5% to about 95%.

The methods of the present disclosure are effective in boosting the immune response, for example, of subjects who are injured, immunocompromised or protein malnourished. Immunocompromised subjects generally exhibit an attenuated or reduced ability to mount a normal cellular or humoral defense to challenge by infectious agents, e.g., viruses, bacteria, fungi and protozoa. Protein malnourished subjects generally have a serum albumin level of less than about 3.2 grams per deciliter (g/dl) and/or unintentional weight loss of greater than 10% of usual body weight.

The methods of the present disclosure can be used to therapeutically or prophylactically treat subjects who are at a heightened risk of infection due to imminent surgery, injury, illness, radiation or chemotherapy, or other condition which deleteriously affects the immune system. The method is useful to treat subjects who have a disease or disorder which causes the normal metabolic immune response to be reduced or depressed, such as HIV infection (AIDS). For example, the method can be used to pre-initiate the metabolic immune response in subjects who are undergoing chemotherapy or radiation therapy, or who are at a heightened risk for developing secondary infections or postoperative complications because of a disease, disorder or treatment resulting in a reduced ability to mobilize the body's normal metabolic responses to infection. Treatment with the compositions of the disclosure has been shown to be particularly effective in mobilizing the host's normal immune defenses, thereby engendering a measure of protection from infection in the treated host.

### Method of Enhancing Immune Response to Vaccination

In some embodiments, the present disclosure provides a generic concept for administering a prebiotic and probiotic bacteria as a therapy for enhancing a subject's immune system or immune response. In some embodiments, administering a prebiotic and probiotic bacteria enhances the immune system thereby providing an advantageous scenario for more effective vaccinations.

In some embodiments, the present disclosure includes methods of enhancing a subject's immune response to vaccination by administering the compositions of the present disclosure, such as an adjuvant. Adjuvant activity is manifested by a significant increase in immune-mediated protection by development of an immune response in an individual who otherwise would not respond at all to a vaccine. Enhancement of humoral immunity is typically manifested by a significant increase in the titer of antibody raised to the antigen.

The methods of the present disclosure, providing the administration of the compositions of the disclosure in conjunction with a vaccine, have the following advantages. The total antigenic load of vaccine to be administered may be reduced since less antigen in the presence of the compositions of the disclosure would elicit an immunologic response at least equivalent to that achieved by the administration of the normal amount of the vaccine. Since less antigen would be required per vaccination by administering the compositions of the disclosure, the probability of undesirable side-effects associated with some vaccines currently in use would be reduced.

The immune response of certain types of individuals who respond poorly to vaccination would be enhanced by administering the compositions of the disclosure in conjunction with a vaccine. Types of individual who should benefit from the methods of the present disclosure include (1) those types having impaired immune responsiveness, (2) those individuals who appear normal but who are nevertheless nonresponsive to certain vaccines as well as (3) individuals undergoing immunosuppressive therapies such as radiation and chemotherapy.

The vaccines contemplated for use in accordance with the present disclosure include but are not limited to bacterial vaccines, toxoid vaccines (inactivated toxins), and viral vaccines, or mixtures thereof used for active immunization. See for example chapter 75 entitled “Immunizing Agents” in Remington's Pharmaceutical Sciences 14th Edition 1990 Mack Publishing Co. p 1426-1441 and the antitoxins, toxoids, vaccines and live vaccines approved by the U.S. Food and Drug Administration and listed on page 208-209 (Product Category Index) of the Physician's Desk Reference, 46th Ed. 1992. Suitable bacterial vaccines include bacterial vaccines against the following disease entities or states: cholera, pertussis, plague, typhoid fever, meningitis, pneumococcal pneumonia, H. influenza type B, leprosy, gonorrhea, Group B meningococcus, and Group B streptococcus, Gram-negative sepsis, E. coli sepsis, and Pseudomonas aeruginosa. Suitable toxoids include diphtheria toxoid, botulism toxic, and tetanus toxoid. Suitable viral vaccines include live and inactivated viral vaccines against the following disease entities or states: poliomyelitis, measles rubella, yellow fever, mumps, hepatitis B, hepatitis C and viral influenza.

In addition, the compositions of the disclosure may be used to enhance the protection afforded by animal or human vaccines that are considered “weak” (i.e., provide diminished protection in terms of level, extent, and/or duration). Examples of such vaccines are bacterins such as Bordetella bacterin, Escherichia coli bacterins, Haemophilus bacterins, Leptospirosis vaccines, Moraxella bovis bacterin, Pasteurella bacterin and Vibrio fetus bacterin, pneumococcal vaccines and attenuated live or killed virus products or recombinant antigenic viral products such as hepatitis B, influenza A & B, bovine respiratory disease vaccine, infectious bovine rhinotracheitis, parainfluenza-3, respiratory syncytial virus, bovine virus diarrhea vaccine, equine influenza vaccine, feline leukemia vaccine, feline respiratory disease vaccine rhinotracheitis pneumonitis caliciviruses, canine parovovirus vaccine, transmissible gastroenteritis vaccine, pseudorabies vaccine, and rabies vaccine.

### Method of Enhancing Immune Response to Treat a Disease or Disorder

In some embodiments, the disclosure is applicable to treating a disease or disorder that is associated with a dysfunctional immune response. In some embodiments, the dysfunctional immune response includes autoimmune diseases. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others. The disclosure should not be limited to only the diseases listed herein. Rather, the disclosure is applicable to any disease associated with dysfunctional immune response.

### Method of Enhancing Immune Response to Cancer

In some embodiments, the disclosure is applicable to tumor vaccines. In some embodiments, the subject has a type of cancer which expresses a tumor-specific antigen. In accordance with the present disclosure, an antigenic composition can be made which comprises a tumor-specific antigen sequence component. In such cases, the combination a tumor-specific antigen is administered in combination with an immunostimulatory agent (e.g., AHCC and B. lactis) to a patient in need thereof, resulting in an improved therapeutic outcome for the patient, evidenced by, e.g., a slowing or diminution of the growth of cancer cells or a solid tumor which expresses the tumor-specific antigen, or a reduction in the total number of cancer cells or total tumor burden.

The present disclosure provides a means to increase immunogenicity of a cell to generate an induced immune response to the tumor-associated antigen in the patient.

In another embodiment, the compounds and combinations of the present disclosure may be used together with existing therapeutic agents used to treat cancer. In some instances, the compounds and combinations of the disclosure may be used together with existing therapeutic agents to activate immune cell function and/or enhance the antitumor effect of the therapeutic agent(s).

In order to evaluate potential therapeutic efficacy of the compounds of the disclosure in combination with the antitumor therapeutics described elsewhere herein, these combinations may be tested for antitumor activity according to methods known in the art.

In one aspect, the present disclosure contemplates that the immunostimulatory agent (e.g., AHCC and B. lactis) of the disclosure may be used in combination with a therapeutic agent such as an anti-tumor agent including but not limited to a chemotherapeutic agent, an anti-cell proliferation agent or any combination thereof.

In one aspect, the present disclosure contemplates that the immunostimulatory agent of the disclosure may be used in combination with a targeted anti-cancer agent, such as monoclonal antibodies, signal transduction inhibitors, gene expression modulators, and the like.

The disclosure should not be limited to any particular chemotherapeutic agent. Rather, any chemotherapeutic agent can be linked to the antibodies of the disclosure. For example, any conventional chemotherapeutic agents of the following non-limiting exemplary classes are included in the disclosure: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

There are a variety of antitumor antibiotics that generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the disclosure—should not be construed as being limited solely to these plant alkaloids.

The taxanes affect cell structures called microtubules that are involved in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the disclosed compositions and methods.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-a, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

### Method of Enhancing Immune Response to Therapy

In some embodiments, the disclosure includes methods of enhancing a subject's immune response to therapy. In various embodiments, the methods of the present disclosure relate to the administration of the disclosed compositions to a subject that is exposed to any number of relevant treatment modalities, including but not limited to treatment with agents such as natalizumab, efalizumab, antiviral agents, chemotherapy, cryotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxin, fludaribine, cyclosporin, FK506, rapamycin, mycophenolic acid, steroids, FR901228, and irradiation.

In this regard, it has been observed that following certain treatments and therapies, in particular treatments and therapies that damage the immune system, shortly after treatment during the period when patients would normally be recovering from the treatment, the quality of the immune system is impaired. Thus, it is contemplated to administer the compositions of the present disclosure to a subject during this recovery phase. The compositions of the present disclosure are especially useful to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In certain embodiments, the methods of the present disclosure comprise the administration of the disclosed compositions to a subject in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the compositions of the disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is involved in growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compositions of the present disclosure are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the compositions of the present disclosure. In an additional embodiment, compositions of the present disclosure are administered before or following surgery.

In another embodiment, the methods of the present disclosure are useful in pain therapy, such as therapy for chronic pain, acute pain, as well as pre-, intra-, and postoperative pain. In various embodiments, the methods of the present disclosure comprise the administration (i.e., pre-administration, co-administration, and/or post-administration) of other treatments and/or agents to modify (e.g., enhance) the effectiveness of the disclosed compositions. In some embodiments, the other agents include anti-pain/anti-inflammation agents.

In some embodiments, the additional anti-pain/anti-inflammation agents may include one or more agents selected from the following classes of receptor antagonists and agonists and enzyme activators and inhibitors, each class. acting through a differing molecular mechanism of action for pain and/or inflammation inhibition: (1) serotonin receptor antagonists; (2) serotonin receptor agonists; (3) histamine receptor antagonists; (4) bradykinin receptor antagonists; (5) kallikrein inhibitors; (6) tachykinin receptor antagonists, including neurokinin, and neurokinin2 receptor subtype antagonists; (7) calcitonin gene-related peptide (CORP) receptor antagonists; (8) interleukin receptor antagonists; (9) inhibitors of enzymes active in the synthetic pathway for arachidonic acid metabolites, including (a) phospholipase inhibitors, including PLA2 isoform inhibitors and PLCγ isoform inhibitors, (b) cyclooxygenase inhibitors, including non-selective cyclooxygenase inhibitors and cyclooxygenase-2 (COX-2) selective inhibitors, and (c) lipoxygenase inhibitors; (10) prostanoid receptor antagonists including eicosanoid EP-1 and EP-4 receptor subtype antagonists and thromboxane receptor subtype antagonists; (11) leukotriene receptor antagonists including leukotriene B4 receptor subtype antagonists and leukotriene D4 receptor subtype antagonists; (12) opioid receptor agonists, including μ-opioid, o-opioid, and K-opioid receptor subtype agonists; (13) purinoceptor agonists and antagonists including P2X receptor antagonists and P2Y receptor agonists; (14) adenosine triphosphate (ATP)-sensitive potassium channel openers; (15) mitogen-activated protein kinase (MAPK) inhibitors; (16) neuronal nicotinic acetylcholine receptor agonists; and (17) soluble receptors. Each of the above agents functions either as an anti-inflammatory agent and/or as an anti-nociceptive, i.e., anti-pain or analgesic, agent.

### Administration

In the various methods of treatment, the administration of the composition(s) disclosed herein may be for either “prophylactic” or “therapeutic” purpose. The composition(s) in accordance with some embodiments are a combination of components, which may be administered separately as individual components of the combination or they may be combined into a single composition, comprising the separate components formulated into a single tablet, capsule, etc. When administered separately, the components may be administered simultaneously, sequentially, or at off-set times.

When provided prophylactically, the composition of the present disclosure is provided in advance of any symptom, although in particular embodiments the vaccine aspect of the disclosure is provided following the onset of one or more symptoms to prevent further symptoms from developing or to prevent present symptoms from becoming worse. The prophylactic administration of composition serves to prevent or ameliorate any subsequent infection or disease. When provided therapeutically, the pharmaceutical composition is provided at or after the onset of a symptom of infection or disease. Thus, the present disclosure may be provided either prior to the anticipated exposure to a disease-causing agent or disease state or after the initiation of the infection or disease.

An effective amount of the composition would be the amount that achieves this selected result of enhancing the immune response, and such an amount could be determined as a matter of routine by a person skilled in the art. For example, an effective amount of for treating an immune system deficiency against cancer or pathogen could be that amount necessary to cause activation of the immune system, resulting in the development of an antigen specific immune response upon exposure to antigen. The term is also synonymous with “sufficient amount.”

In order to impart their prophylactic and/or therapeutic effect, the disclosed combination (e.g., a prebiotic component, such as a mushroom extract, including but not limited to AHCC or K1), and a probiotic component (e.g., a microbial cell or culture, living or attenuated, or cellular fractions thereof, including cell walls and metabolites) may be delivered to a subject by any conventional delivery means. In some embodiments, the subject is a mammal. In another embodiment, the subject is a cell. One mechanism for delivery is by any of the methods mentioned above which physically or chemically permeabilize the cell membrane. Another embodiment of the disclosure for transferring the prophylactic and/or therapeutic combination into cells may involve particle bombardment. This method depends on the ability to accelerate microprojectiles carrying the short chain fatty acids to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In a further embodiment, the compositions may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

In another embodiment, the compositions may be immobilized on the surface of a substrate. The substrate surface may be any surface capable of having an agent/ligand bound thereto or integrated into and that is biocompatible. The biocompatible surface may be biodegradable or non-biodegradable. The surface may be natural or synthetic, and a synthetic surface may be a polymer. The surface may comprise collagen, purified proteins, purified peptides, polysaccharides, glycosaminoglycans, or extracellular matrix compositions. A polysaccharide may include for example, cellulose, agarose, dextran, chitosan, hyaluronic acid, or alginate. Other polymers may include polyesters, polyethers, polyanhydrides, polyalkylcyanoacryllates, polyacrylamides, polyorthoesters, polyphosphazenes, polyvinylacetates, block copolymers, polypropylene, polytetrafluorethylene (PTFE), or polyurethanes. The polymer may be lactic acid or a copolymer. A copolymer may comprise lactic acid and glycolic acid (PLGA). Non-biodegradable surfaces may include polymers, such as poly(dimethylsiloxane) and poly(ethylene-vinyl acetate). Biocompatible surfaces include for example, glass (e.g., bioglass), collagen, metal, hydroxyapatite, aluminate, bioceramic materials, hyaluronic acid polymers, alginate, acrylic ester polymers, lactic acid polymer, glycolic acid polymer, lactic acid/glycolic acid polymer, purified proteins, purified peptides, or extracellular matrix compositions. Other polymers comprising a surface may include glass, silica, silicon, hydroxyapatite, hydrogels, collagen, acrolein, polyacrylamide, polypropylene, polystyrene, nylon, or any number of plastics or synthetic organic polymers, or the like.

In some embodiments, the compositions may be formulated in a form suitable for delivery to a subject having a condition in need of treatment or a condition at risk of development in need of prevention. In some embodiments, the subject is a human. In some embodiments, the subject is a non-human. In some embodiments, the disclosed methods of producing the compositions further comprise formulating the compositions into a form suitable for delivery to a human and a non-human.

In some embodiments, the compositions are formulated in a form suitable for delivery as a dietary supplement, a nutraceutical, a medical food, an animal feedstuff, and/or a nasal spray. In some embodiments, the method further comprises formulating the compositions into a form suitable for delivery via one or more routes of administration.

Non-limiting examples of routes of administration include parenteral, subcutaneous, intravascular injection or infusion, intramuscular injection, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

In some embodiments, the compositions are formulated within one or more suppositories. In some embodiments, the one or more suppositories comprise active ingredients, inactive ingredients, excipients, additives, and/or pharmaceutically acceptable carriers. Non-limiting examples of additives include natural polymer compounds, inorganic salts, binders, lubricants, disintegrants, surfactants, thickeners, coating agents, pH adjusters, antioxidants, flavoring agents, preservatives, and colorants among others. Non-limiting examples of other pharmaceutically acceptable carriers include liquid carriers such as water, alcohol, emulsion, and solid carriers such as gel, powder, and the like.

In some embodiments, the compositions are formulated for intravenous administration with excipients and pharmaceutically acceptable carries including one or more of sodium chloride, dextrose, and sterile water, for example, in the form of aqueous isotonic sterile injection solutions, comprising one or more of antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some embodiments, the compositions are formulated for administration by intravenous infusion such as injection solutions and suspensions prepared from sterile powders, granules, and/or tablets in unit-dose or multi-dose in sealed containers such as ampules and/or vials.

In some embodiments, the compositions are formulated in the form of a dietary supplement tablet or capsule. In some embodiments, the method further comprises formulating the compositions with other components to be used as a nutraceutical or medical food for human consumption or formulated with animal feed material as a substitute for antibiotics. Examples including formulating the compositions with dietary supplements, food additives, nutrients, micronutrients, vitamins, minerals, additional active agents, as well as conventional excipients used in oral delivery formulations.

The components of the combination may be administered orally, and thus be formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. If formulated in form of a capsule, the compositions of the present disclosure comprise, in addition to the active compound and the inert carrier or diluent, a hard gelating capsule. In some embodiments, the dried product is formulated for oral administration in any dosage form that is suitable for oral ingestion. Non-limiting examples include liquid compositions such as elixir, suspension, syrup, emulsion, ampoule, etc., solid compositions such as gel, gum, drop, powder, granule, pill, sugar-coated tablet, film-coated tablet, capsule, package agent, sustained-release compositions such as gel-coated compositions, multi-coated compositions, localized release compositions, and the like.

The components of the combination may be further administered intranasally, i.e. by inhalation and thus may be formulated in a form suitable for intranasal administration, i.e. as an aerosol or a liquid preparation. In some embodiments, the method further comprises formulating the dried product for nasal administration in any dosage form that is suitable for nasal delivery. Non-limiting examples include nasal spray, nasal drops, metered dose inhalers, aerosols, and the like. Nasal delivery formulations and methods may be useful in treating and/or preventing common cold, influenza, and allergies.

The compositions may also, for example, be formulated as suppositories, containing conventional suppository bases for use in human or veterinary medicine or as pessaries, for example, containing conventional pessary bases.

The compositions may, for example, be formulated for parenteral administration (e.g., by injection, for example, bolus injection or continuous infusion) and may be presented in unit dose form in ampules, pre-filled syringes, small volume infusion containers or in multi-dose containers with an added preservative. The injection formulations may be suitable for intradermal, subcutaneous, intramuscular, or intravenous injection. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

The amount, frequency and period of administration will vary depending upon factors such as the level of the specific antibody titers, the class of antibody to be induced, the vaccine type as well as the age of the patient and general physical condition. The compositions of the disclosure can be administered before, concurrently with, or after the vaccine is administered.

In some embodiments, the compositions are administered separately from the vaccine, although it may be administered in combination with the vaccine. For instance, when the compositions are combined with the vaccine, the composition administered may contain an immunogen that is effective in eliciting a specific response to a given pathogen or antigen, a pharmaceutically acceptable vaccine carrier, and an immunopotentiating amount of the compositions of the disclosure. In some embodiments, the compositions of the disclosure are administered prior to the administration of the vaccine and at the same site where the vaccine is to be administered. The formulations and pharmaceutical compositions contemplated by the above dosage forms can be prepared with conventional pharmaceutically acceptable excipients and additives, using conventional techniques. Other adjuvants may be administered either with the vaccine or together with the compositions of the disclosure.

If multiple doses of the vaccine are to be administered over a period of time, additional doses of the compositions may be administered in conjunction with each subsequent dose of the vaccine. The amount of the compositions of the disclosure so administered with each subsequent dose of the vaccine may be more, the same or less than the amount of the compositions administered in conjunction with the initial dose of the vaccine. The amount of the compositions of the disclosure administered with each subsequent dose of the vaccine will depend upon the antibody response of the patient after the first dose of the vaccine.

In various embodiments, the immunostimulatory agent(s) may be co-administered with various other compounds (cytokines, chemotherapeutic and/or antiviral drugs, among many others). Alternatively, the immunostimulatory agent(s) may be administered an hour, a day, a week, a month, or even more, in advance of an immunogenic composition, or any permutation thereof. Further, the immunostimulatory agent may be administered an hour, a day, a week, or even more, after administration of an immunogenic composition, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various immunostimulatory agent and the immunogenic composition, and the like.

In some embodiments, one or more of the following routes of administration are contemplated: parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal. In some embodiments, the composition to be administered can be formulated for delivery via one or more of the above noted routes.

Any order of administration can be used for the prebiotic and the probiotic in a combination. For example, the one or more prebiotics and the one or more probiotics can be administered simultaneously or sequentially. For example, all components of the combination are administered simultaneously, or only some of the components of the combination are administered simultaneously and the rest are administered sequentially. In some embodiments, none of the components are administered simultaneously, i.e., all the components are administered sequentially. When administering sequentially, any order of administration can be used.

In some embodiments, a frequency of administration of the prebiotic and/or probiotic can be varied depending various parameters such as level of potentiation, prognosis following administration of a combination provided herein, patient compliance, side effects, etc., for example, daily, weekly, biweekly, monthly, bimonthly. Prebiotics can be administered along with probiotics daily, weekly, biweekly, monthly, bimonthly. In some embodiments, the prebiotic is administered less frequently compared to the probiotic, or more frequently compared to the probiotic.

In some embodiments, administration can be daily, or 1, 2, 3, 4, 5, 6 or more times weekly, or more or less frequently as required. In some embodiments, administration can be provided as a single dose or as divided doses, such that a daily dose may be given in 2, 3, 4, or more portions in a single day.

In some embodiments, co-administration of the components of a combination may comprise administering the components simultaneously, or within about 1, 5, 15, 30, 45 or 60 minute of one another, within about 1 hour to within about 6 hours of one another, or as desired by one of ordinary skill in the art.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

It is believed that one of ordinary skill in the art, based on the preceding description and the following illustrative examples, can make and utilize the compounds of the present disclosure and practice the claimed methods. Therefore, the following working examples refer to some embodiments of the present disclosure, and are not to be construed as limiting in any way.

### Example 1—Effects of a Nutraceutical Blend on Selected Immune Parameters

A randomized, double-blinded, placebo controlled study was conducted, in which 52 healthy subjects ingested either a placebo or a composition comprising AHCC (1,800 mg AHCC-FD/day) with a probiotic product B. lactis (12 billion CFU/day) for 8 weeks. At the 4 week time point, the subjects were administered a flu vaccine. The ingredients of the composition were blended, then encapsulated. A color-matched placebo was made using inert coloring substances and the same excipients as used in the active product. Blood was drawn at the 0, 4, and 8 week time points. The blood was analyzed for several parameters including red blood cell numbers, hemoglobin content, hematocrit, and numbers of various subsets of circulating immune cells at the various time points.

Distinct changes were found in the numbers of various subsets of circulating immune cells during the consumption of AHCC/B. lactis in the absence of an immune challenge (loading phase; Day 0 (D0)-Day 28 (D28)), as well as after the vaccine challenge (D28-Day 56 (D56)). General observations throughout the study include: a significant improvement in the red blood cell numbers, hemoglobin content, and hematocrit in the female population (FIG. 1); and a continuous increase in CD11c+ myeloid dendritic cells (FIG. 2) and CD14+ CD16+ monocytes (FIG. 4) throughout the 8-week study.

Observations made during the loading phase include: an increase in the numbers of myeloid dendritic cells; and an increase in CD14+ CD16+ monocytes (FIGS. 2 and 4). These changes may have led to more effective antigen presentation during the vaccine challenge, thus preparing the immune system for an immune challenge.

Observations made after the vaccine challenge include: a continued increase in CD14+ monocytes (FIG. 3); a continued increase in CD14+ CD16+ monocytes (FIG. 4); and a specific increase in the IgG3 subclass of vaccine-specific IgG (FIG. 5). Data suggests a protection from the reduction in several cell types otherwise seen after the vaccine challenge: CD56++ NK cells, CD4+ T cells, and CD8+ T cells.

Following the vaccine challenge, several effector cell types were increased in the blood circulation in the AHCC/B. lactis group compared to the placebo group, suggestive of an improved alertness of the immune system. In addition, the reduction in multiple cell types seen after the vaccine challenge in the placebo group was attenuated in the group consuming AHCC/B. lactis.

The multitude of phenotypic data and numbers of immune cell subsets presented here only represents one of several aspects of what can be measured in the blood circulation, pertaining to immune function. Several other aspects of the immune status are relevant, and material has been banked so that such testing can be performed at a later time, without repeating the clinical phase, but allowing direct correlations to the existing data sets.

The disclosures of each and every patent, patent application, and publication cited herein are hereby incorporated herein by reference in their entirety.

### Example 2—Protective Activity of Combination of AHCC and UREX Against Murine Vaginal Candidiasis

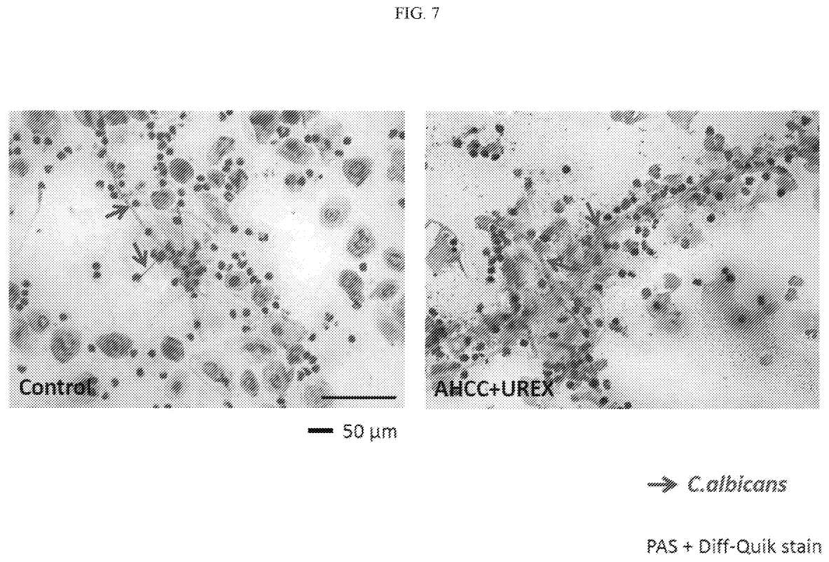

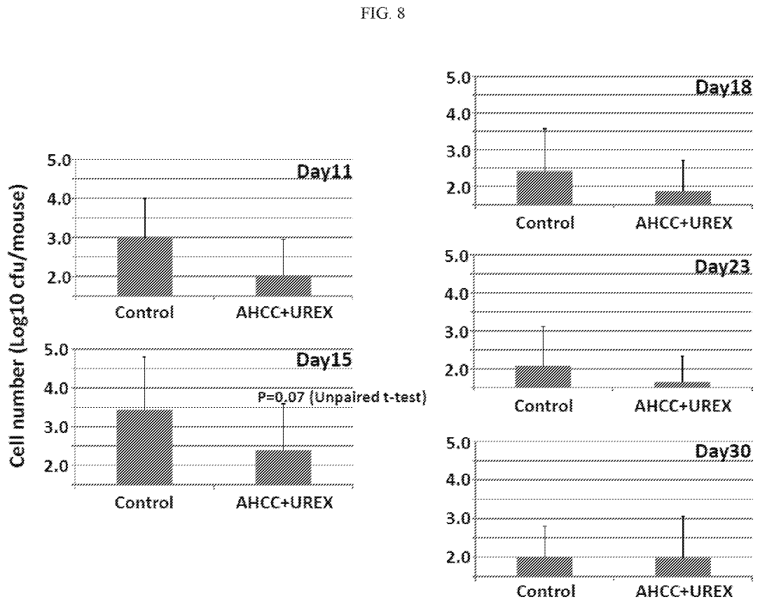

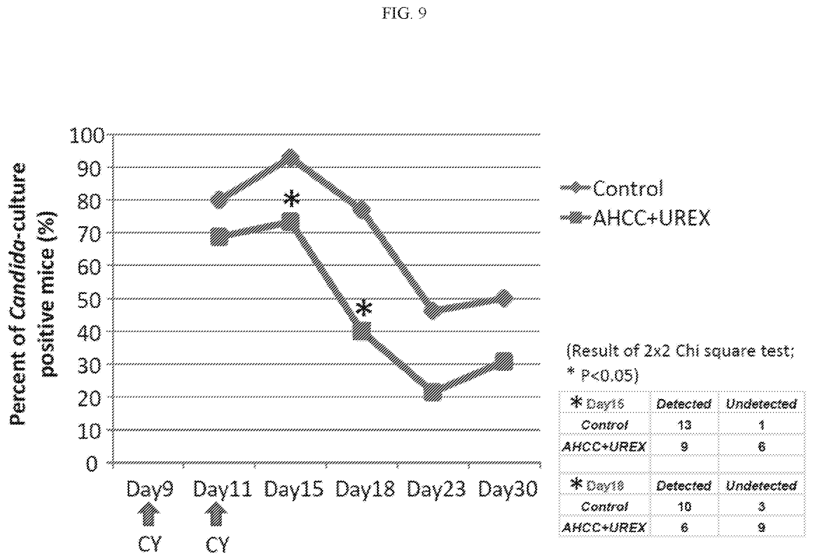

Oral administration of the combination of AHCC and UREX was suggested to have protective activity against murine vaginal candidiasis. Vaginal candidiasis, caused by Candida species, is a common fungal infection for the immune-suppressed people, diabetic patients and pregnant women. Recent years, it was suggested that AHCC may display a protective role against opportunistic fungal infection containing Candida albicans in leukopenic hosts, and that UREX, a mixture of Lactobacillus rhamnosus GR-1 and Lactobacillus reuteri RC-14, can increase the effectiveness of an anti-fungal pharmaceutical agent in curing vulvovaginal candidiasis. Thus, protective activity of combination of AHCC and UREX was examined using Candida vaginitis model. Experimental schedule of murine vaginal Candidiasis is shown in FIG. 6. Hyphal candida cells as pathogenic growth forms were observed microscopically from vaginal cavities (FIG. 7). As seen in FIG. 8, oral administration of AHCC and UREX appeared to decrease the number of the viable C. albicans cells in the vaginal cavity. Oral intake of AHCC and UREX facilitated the exclusion of Candida cells from vaginal cavities of the mice with vaginal candidiasis (FIG. 9). At the final day of the experiment (Day 30), the number of Candida cell in kidneys of residual mice was examined. No viable C. albicans was detected.

These results suggest that intake of AHCC plus UREX decreased the number of viable Candida cells in vaginal cavities of the infected mice and facilitated exclusion of Candida cells from the vaginal cavities. All mice were intraperitoneally treated with cyclophosphamide (50 mg/kg×2) to induce neutropenia. Oral administration of AHCC was reported to facilitate the recovery of the mice from neutropenia. Therefore, without being bound by any theory, the mechanisms in protective activity of AHCC may be explained by them. By clinical study, oral intake of UREX plus fluconazole was reported to have therapeutic efficacy. These results suggest that therapeutic effect of AHCC plus UREX also will be applicable to clinical vaginal Candidiasis.

### Example 3

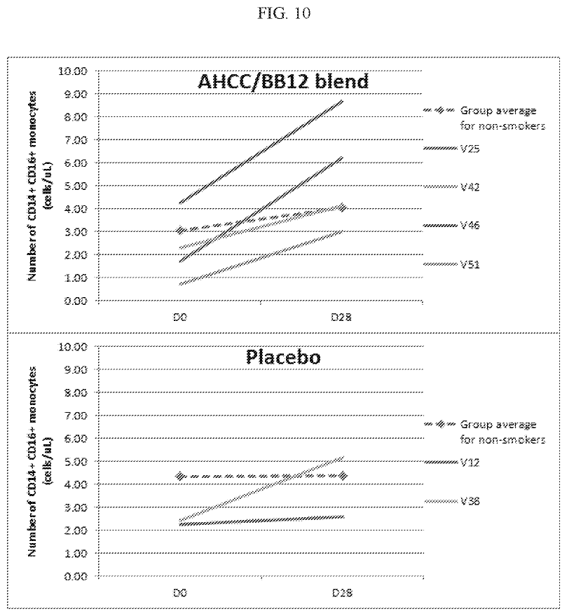

Data comparing smokers versus non-smokers on AHCC/B. lactis blend and placebo are shown in FIG. 10. Smokers are represented in the graphs as “V.” Data are shown for analysis over the 4-week loading phase of the AHCC/B. lactis blend, not the post-loading response. Without being bound by any theory, this was based on the rationale that a 4 week consumption of AHCC/B. lactis may not be sufficient for all smokers to overcome their smoking-induced immune suppression.

Evaluation of group averages of non-smokers in each group and comparison to smokers indicated that most smokers started the study with monocyte levels that were lower than the non-smokers, suggesting that this cell type is depleted in many smokers (FIG. 10).

As shown by data in FIG. 10, a clear trend was observed for CD14+CD16+ monocytes, cells that are involved in phagocytosis, antigen-presentation, cytokine production, and immune regulation.

For AHCC/B. lactis blend, the non-smokers showed a mild, but significant increase in the numbers of this type of monocytes after 4 weeks consumption (dashed line) whereas all four smokers showed an increase that was more robust than the average increase seen in the non-smokers. For placebo, the group average did not change during the 4 weeks. The number of monocytes for the one smoker in this group (V12) did not change (bottom line). The number of monocytes increased in the one volunteer in the placebo group (V38) that had quit smoking just before study start (FIG. 10). This may represent the volunteer's immune system reverting back to normal since the volunteer stopped smoking.

### Example 4

A group of patients with cancer are selected. Prior to administration of a composition comprising prebiotic and probiotic according to the present disclosure laboratory tests are performed to obtain baseline cytokine production, NK cell activity, macrophage numbers, dendritic cell numbers, and T cell numbers. The group is split into subgroups. One subgroup is administered a composition comprising prebiotic and probiotic (test composition 1), another subgroup is administered a composition comprising prebiotic (test composition 2), a third subgroup is administered a composition comprising a probiotic (test composition 3), and a fourth subgroup is administered placebo.

After about 30 days of administration of test compositions, a second round of laboratory tests are performed to compare cytokine production, NK cell activity, macrophage numbers, dendritic cell numbers, and T cell numbers in the test and placebo subgroups.

Similarly, after about 60 days of administration of either the test compositions, a second round of laboratory tests are performed to compare cytokine production, NK cell activity, macrophage numbers, dendritic cell numbers, and T cell numbers in the test and placebo subgroups.

Laboratory tests on days 30 and 60 show significant increases in cytokine production, NK cell activity, macrophage numbers, dendritic cell numbers, and T cell numbers in the test subgroups as compared to placebo subgroup suggesting an increase in innate and adaptive immune responses in test subgroups. Additionally, significant reduction in cancer is observed in the test subgroups as compared to the placebo subgroup.

Furthermore, the increases in cytokine production, NK cell activity, macrophage numbers, dendritic cell numbers, and T cell numbers, and reduction in cancer in the test subgroup that is administered test composition 1 is greater than the sum of the increases in cytokine production, NK cell activity, macrophage numbers, dendritic cell numbers, and T cell numbers, and reduction in cancer observed in test subgroups administered either test composition 2 alone or test composition 3 alone. Thus, a synergistic effect is observed when a combination of prebiotic and probiotic used.

### Example 5—Production of K1

The solid substrate medium used was hydrated cooked grain. The preferred grain is rice, oats, wheat, rye or millet. The grain was pre-soaked in water and heated to 100° C. for 30 min-60 min. While heating, additional carbohydrates can be added that include glucose, maltose or sucrose along with nitrogen supplements such as soy protein, dried yeast, or peptone. Excess water was removed by draining or decanting and moisten grain is added to autoclavable containers either polypropylene jars or polypropylene bags both of which have filters to allow gas exchange. The filled containers were autoclaved at 121° C. and 15-18 psi for 60 min. The sterilized containers were cooled to room temperature and aseptically inoculated with appropriated strains of fungi. The inoculated jars or bags were subsequently sealed and incubated in the dark for a period between 35 d-60 d at a temperature of preferably 16° C.-21° C. and a RH of 70%.

K1 was produced using a mix of six different cultivars (LE-1 through LE-6) of Lentinula edodes cultivated on solid substrate of rice and wheat for 60 d.

After cultivation (Example 2), the grain colonized mycelium was removed from the container and dried at 70° C. overnight then ground into a fine powder. The extraction involved heating 500 ml water to between 80° C.-90° C. and adding 50 g of the dried powder. The mixture was covered and heated with continuous agitation for a period of 2 hr, at which time, 2 grams amylase was added to the mixture, and extraction continued for another 1 hr. The slurry was centrifuged at 4,500 g for 10 min to remove debris, the supernatant decanted and freeze dried to obtain the powdered K1 product. The yield for K1 averaged 10 g/50 g dry weight starting material.

K1 a slightly pale ochre powder with the following composition:

| | | | | Range α | Range β |
|---|---|---|---|---|---|
| Total sugar | Protein | Lipid | Other | glucans | glucans |
| 81.5 g/100 g | 3.4 h/100 g | 0.7 g/100 g | 14.4 g/100 g | 9.9%-25.0% | 10.9%-30.2% |

Average molecular weight determination of polysaccharide fraction obtained from Sephadex gel filtration chromatography for K1: Less than 1,500 Da=13.2%; Between 1,500 Da-5,000 Da=10.3%; Between 5,000 Da-10,000 Da=25.0%; Greater than 10,000 Da=51.5%. Primary sugar as determined by paper chromatography and NMR analysis is glucose for KI.

Characteristic retention time fingerprint and % composition of de-lipidized sample by high performance liquid chromatography utilizing Agilent Hi-Plex Na column with water elution 0.300 ml/min. at 85° C. and a pressure of 25 bar, with UV detection (at 280 nm).

| Sample | RT | % Area |
|---|---|---|
| K1 | 10.505 | 32.6 |
| | 11.246 | 29.2 |
| | 24.009 | 38.2 |

Major peaks of K1 as shown for ¹H and ¹³C NMR spectrum:

| Sample | Component | ¹H NMR | ¹³C NMR |
|---|---|---|---|
| K1 | Aliphatic | 0.9-1.0, 1.5, 2.1, | |
| | | 2.4, 2.6-2.7 ppm | |
| | Sugars | 3.2, 3.4-3.6, | 62-65, 70-75, |
| | | 3.7-3.9 ppm | 76-80, 95, 97, |
| | | | 101, 106 ppm |
| K1 | B glucose | 4.6-4.7 ppm | |
| K1 | α glucose | 5.2 ppm | |
| K1 | α 1-4 | 5.4 ppm | |
| | glucose | | |

### Example 6—K1 and Immune Activation

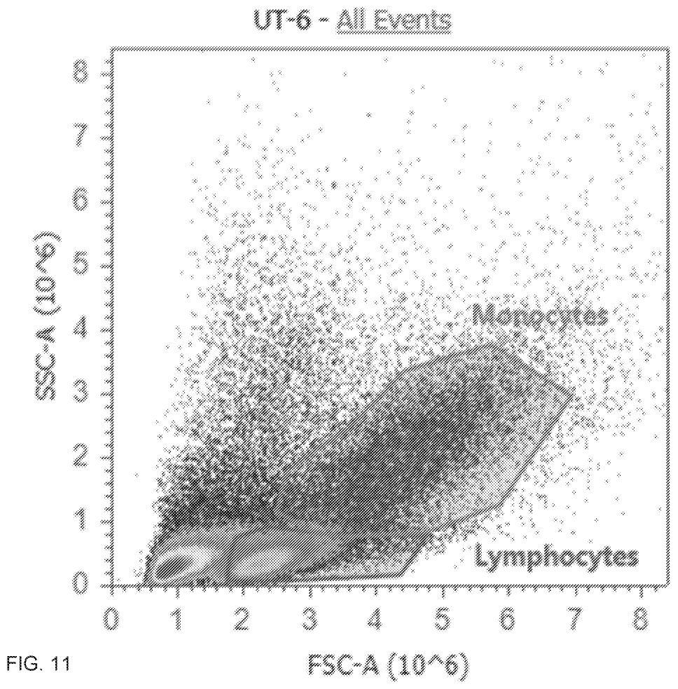

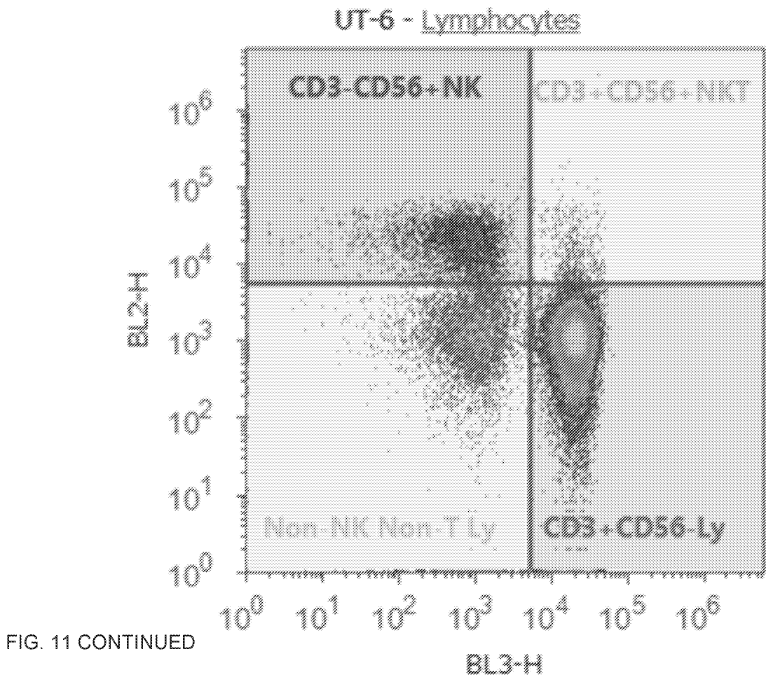

K1 and commercially available AHCC were compared for immune activating properties. Freshly isolated human peripheral blood mononuclear cell (PBMC) from healthy human donors were used for this testing, following published protocols from NIS Labs. A set of cultures were left untreated as negative control cultures for immune activation. Triplicate sets of cultures were treated with serial dilutions of the test product. The inflammatory bacterial lipopolysaccharide LPS from E. coli was used as a positive control for activation. The cultures were incubated for 24 hours, after which the cells and the culture supernatants were harvested and used to monitor the reactions in each culture. Immunostaining was performed to document expression levels of CD69. FIG. 11 shows flow cytometry data showing gates for lymphocytes, monocytes, and the four subsets of lymphocytes, allowing analysis of CD69 expression on all five cell types.

As shown in FIG. 11, the earliest inducible cell surface glycoprotein during lymphoid activation resulting in lymphocyte proliferation and cellular signaling, and an integral part of the mechanisms involved in NK cell activity against target cells.

While CD69 plays a role in immunity through the increase of lymphocyte proliferation and cellular signaling, it has also been implicated in the immunomodulatory effects leading to the control of inflammation. When human NK cells are co-cultured with target cells, CD69 expression is upregulated, and the increase significantly correlated with NK cell secretory activity, as measured by the gold-standard CD107 mobilization assay.

A direct and highly significant correlation between CD69 levels and NK cell cytotoxic activity was demonstrated by Clausen et al 2003, in a study involving 14 breast cancer patients tested repeatedly during chemotherapy. Therefore, CD69 is of key importance for studying NK cells. It has been used for the study of natural products, including enzymatically modified rice bran (MGN-3), extracts from Reishi (Ganoderma lucidum), and extracts from Echinacea and Astragalus. CD69 staining for NK cell activation, indicative of NK cell functional status, was applied to a number of published studies over the past 12 years, both in vitro and in clinical studies.

K1 was more robust than AHCC on multiple aspects of immune cell activation, including the expression of CD69 on lymphocytes and monocytes and activation of NK cells. FIG. 12 shows the effects K1 and AHCC on NK cell activation. The percent change in CD69 expression from the untreated control cultures are shown as the average±standard deviation for each data set.

As shown in FIG. 12, the immune cell activation mediated by K1 surpassed that of AHCC. AHCC only induced moderate activation of monocytes/macrophages, and AHCC did not activate NK cells.

### Example 7—K1 and Cytokine Induction

K1 treatment of immune cells revealed a much more complex effect on cytokine profile than AHCC. It was especially noteworthy that K1 showed robust effects on upregulation of cytokines traditionally associated with immune activation, while also showing very potent effects on induction of anti-inflammatory mediators playing key roles in the resolution of inflammation.

Effects of K1 and AHCC in reducing expression of inflammatory cytokines is shown in FIG. 13, and the effects of K1 and AHCC on immune-activating, pro-inflammatory cytokines and chemokines is shown in FIG. 14. The effects K1 and AHCC on immune-activating pro-inflammatory cytokines and chemokines in human cell cultures are shown in FIG. 13. The percent change in cytokine levels from the untreated control cultures are shown as the average±standard deviation for each data set. FIG. 14 shows the effects K1 and AHCC on anti-inflammatory cytokines. The percent change in cytokine levels from the untreated control cultures are shown as the average±standard deviation for each data set. The levels of cytokines secreted by the immune cell cultures were determined using Luminex multiplex protein arrays and the MagPix platform that allows for simultaneous testing of all analytes in the same samples of culture supernatants.

### Example 8—K1: Effects on Breast Cancer Cell Line

The direct tumor growth-inhibiting effects of K1 were tested on the well-published breast cancer epithelial cell line MCF-7. This cell line grows in vitro in a manner that resembles the epithelial milk ducts in the human breast, and if cultured in 3-D cultures can form complete spheres secreting milk proteins into the center of the sphere. It can be manipulated in various ways to lose this structure and grow in disorganized masses of cells.

FIG. 15 shows the effect of K1 and AHCC (1 mg/mL) on the MCF-7 breast cancer cell line is shown as a result of the conversion of MTT reagent to the colored compound Formazan by the mitochondrial reductase enzyme in viable cells. Data in FIG. 15 show that testing revealed evidence for tumor cell growth-reducing effects for K1, with a robust effect at higher doses, reaching a high level of statistical significance, both when compared to untreated MCF-7 cultures, and to MCF-7 cultures treated with AHCC. This particular data is of relevance for clinical pilot work currently being initiated in a small study of breast cancer patients. K1 triggered reduced cell viability, as reflected by a reduced colorimetric reaction in the MTT assay. This effect was highly significant when compared to untreated cultures and AHCC-treated cultures.

The results highlight K1 as a multifaceted ingredient with complex immune activating, anti-inflammatory, and effector-enhancing activities, of promise for regulation and resolution of inflammation. Further, the results showed superiority of K1 above AHCC, both in terms of immune cell activation and induction of cytokines.

### Example 9—K1 and Probiotic Cell Wall Fraction: Immune Activation

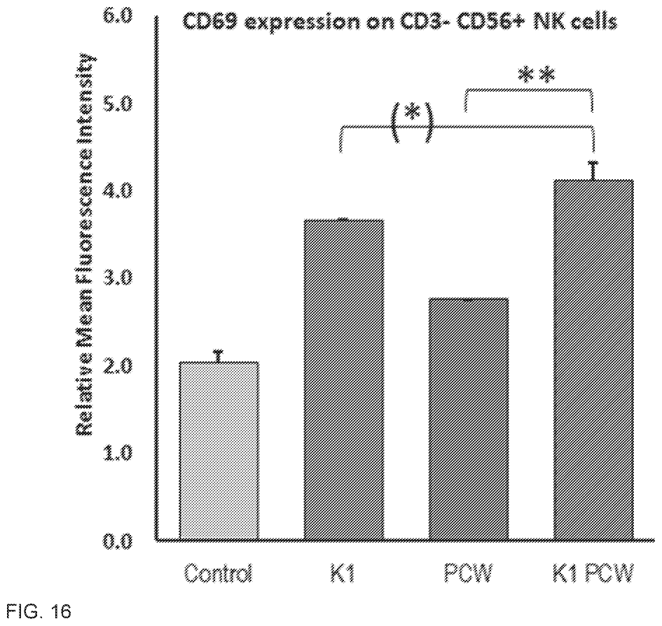

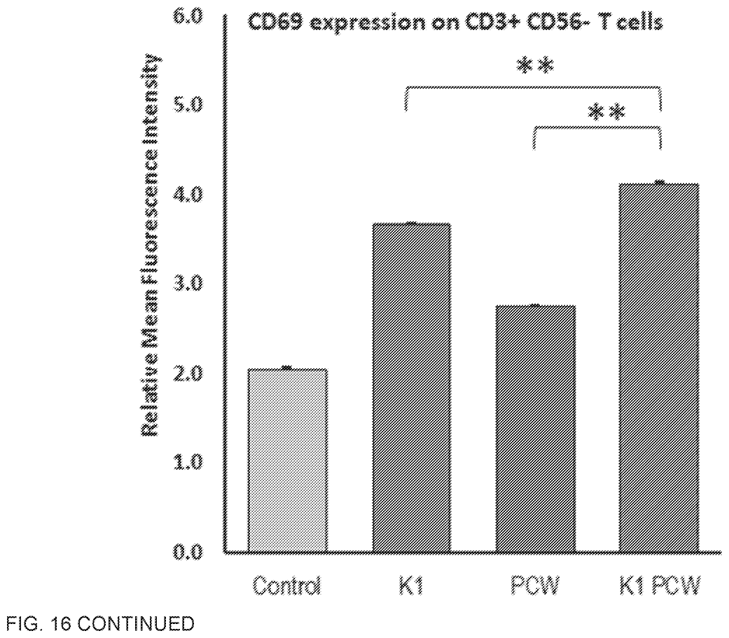

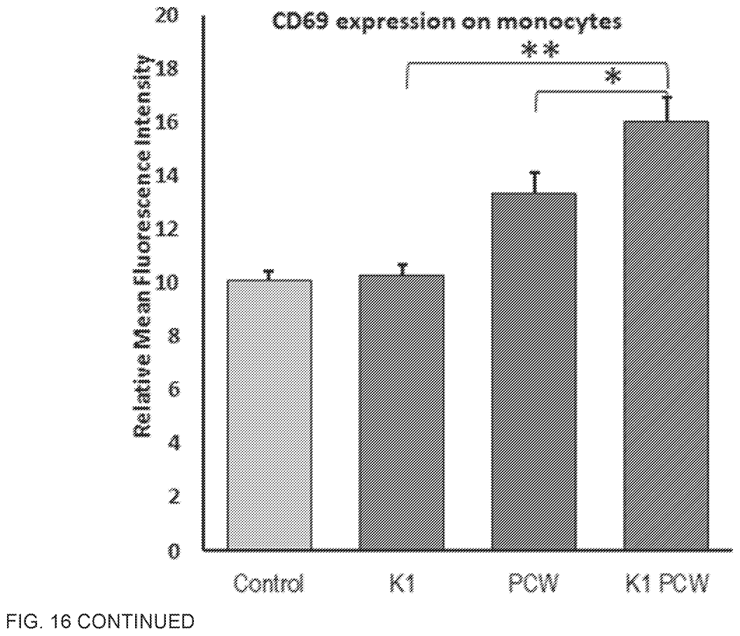

K1, the probiotic cell wall fraction, and the blend thereof were compared for immune activating properties. Freshly isolated human peripheral blood mononuclear cell (PBMC) from healthy human donors were used for this testing, following published protocols from NIS Labs. A set of cultures was left untreated as a negative control for immune activation. Triplicate sets of cultures were treated with serial dilutions of the test products. The cultures were incubated for 24 hours, after which the cells and the culture supernatants were harvested and used to monitor activation in each culture. Immunostaining was performed to document expression levels of CD69, the earliest inducible cell surface glycoprotein during lymphoid activation resulting in lymphocyte proliferation and cellular signaling, and an integral part of mechanisms involved in Natural Killer cell activity against target cells. Flow cytometry analyses with gates for lymphocytes, monocytes, and the four subsets of lymphocytes, allowed analysis of CD69 expression on all five cell types, as shown in FIG. 11. The effects K1 (0.2 mg/mL), the probiotic cell wall fraction (PCW), and the blend thereof (K1 PCW) on NK cell activation are shown in FIG. 16. The mean fluorescence intensity of CD69 expression are shown as the average±standard deviation for each data set.

CD69 plays a role in immunity through the increase of lymphocyte proliferation and cellular signaling. It has also been implicated in the immunomodulatory effects leading to the control of inflammation. When human NK cells are co-cultured with target cells, CD69 expression is upregulated, and the increase significantly correlated with NK cell secretory activity, as measured by the gold-standard CD107 mobilization assay. A direct and highly significant correlation between CD69 levels and NK cell cytotoxic activity was demonstrated by Clausen et al 2003, in a study involving 14 breast cancer patients tested repeatedly during chemotherapy. Therefore, CD69 is of key importance for studying NK cells. It has been used for the study of natural products, including enzymatically modified rice bran (MGN-3), extracts from Reishi (Ganoderma lucidum), and extracts from Echinacea and Astragalus. CD69 staining for NK cell activation, indicative of NK cell functional status, was applied to a number of published studies over the past 12 years, both in vitro and in clinical studies.

Both K1 and probiotic cell wall fraction (“PCW”) showed immune cell activation, and effects included the increased expression of CD69 on Natural Killer cell, T lymphocytes, and monocytes. The immune cell activation mediated by the K1 PCW blend surpassed that of each ingredient alone.

### Example 10—Synergy Between K1 and Probiotic Metabolites and Cell Wall Fractions: Effects on Cytokine Induction

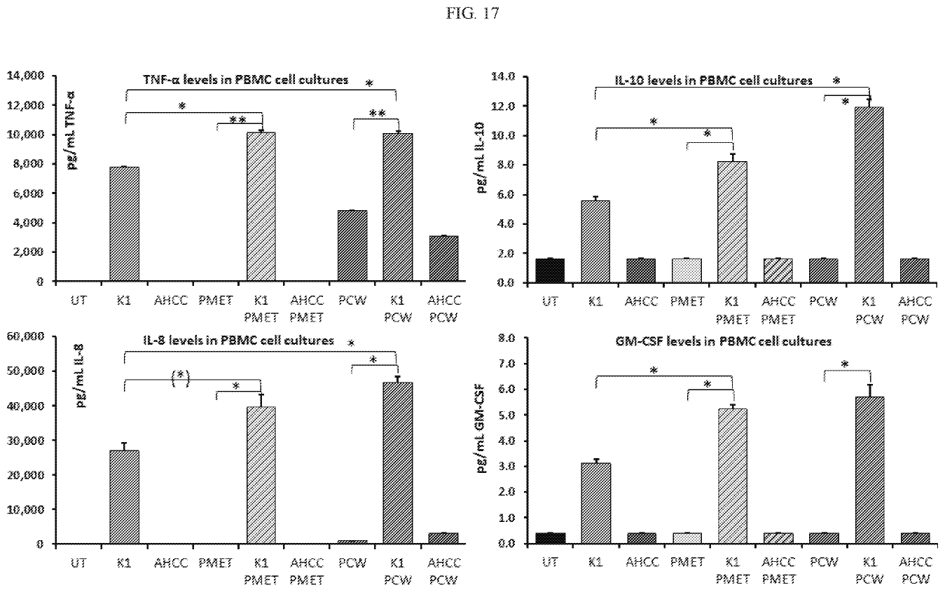

K1 treatment of immune cells revealed a much more complex effect on cytokine profile than AHCC. FIG. 17 shows the effects K1 and AHCC, alone and blended with probiotic metabolites (PMET) versus probiotic cell walls (PCW) on the immune-activating pro-inflammatory cytokines TNF-α and IL-8, the anti-inflammatory cytokine IL-10, and the stem cell growth factor G-CSF in human immune cell cultures. The percent change in cytokine levels from the untreated control cultures are shown as the average±standard deviation for each data set. Statistical significance levels are indicated for the comparison of K1 alone versus blended with probiotic fractions, where P<0.05 is indicated by a single asterisk *, and a high level of significance P<0.01 is indicated by a double asterisk **. As shown in FIG. 17, it was especially noteworthy that K1 showed robust effects on upregulation of cytokines traditionally associated with immune activation, while also showing very potent effects on induction of anti-inflammatory mediators playing key roles in the resolution of inflammation, such as IL-10.

Despite the lack of cytokine induction by the probiotic metabolites alone, the blend containing K1 and probiotic metabolites showed significantly higher cytokine induction than for K1 alone. This suggests a unique and potent, synergistic enhancement of the K1-mediated immune modulating effects by probiotic metabolites, secreted by live probiotic bacteria.

The effects of the blend were significantly higher than K1 alone for the anti-inflammatory cytokine IL-10 as well as for the two biomarkers involved in stem cell biology namely IL-8 and G-CSF, and reached a high level of significance for TNF-alpha.

### Example 11—K1 Blended with Probiotic Cell Walls: Effects on Breast Cancer Cell Line

The direct tumor growth-inhibiting effects of K1 alone and blended with probiotic cell walls were tested on the well-published breast cancer epithelial cell line MCF-7. This cell line grows in vitro in a manner that resembles the epithelial milk ducts in the human breast, and if cultured in 3-D cultures can form complete spheres secreting milk proteins into the center of the sphere. It can be manipulated in various ways to lose this structure and grow in disorganized masses of cells.

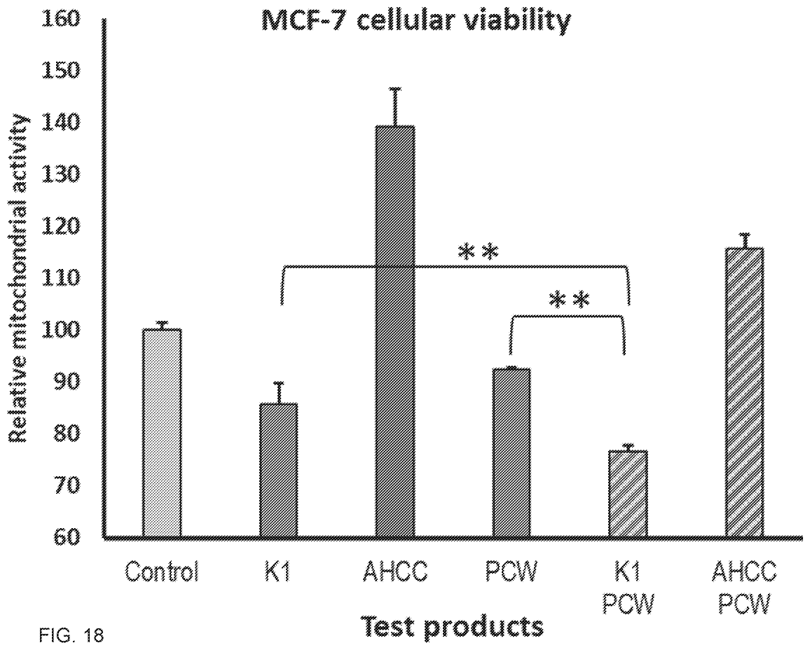

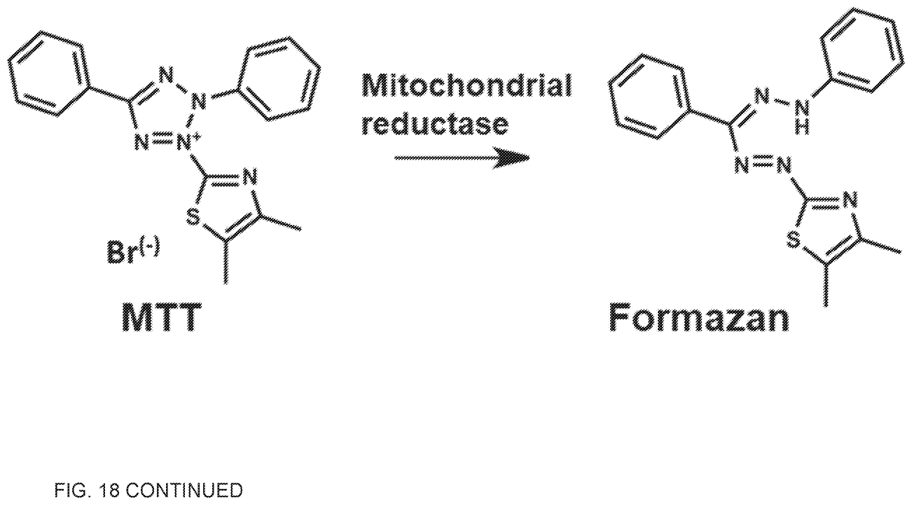

FIG. 18 shows the effect of K1 and AHCC (1 mg/mL) on the MCF-7 breast cancer cell line is compared to the effects of probiotic cell walls (PCW), as well as blends of K1/PCW and AHCC/PCW. The data reflect the conversion of MTT reagent to the colored compound Formazan by the mitochondrial reductase enzyme in viable cells. Both K1 and PCW triggered reduced cell viability, as reflected by a reduced colorimetric reaction in the MTT assay, when compared to untreated control cultures. The K1/PCW blend triggered more robust reduction of MCF-7 viability than either ingredient alone. The difference between K1 or PCW alone, versus the K1/PCW blend was highly significant when compared to untreated cultures and AHCC-treated cultures (P<0.01, **). As shown in FIG. 18, the testing revealed evidence for tumor cell growth-reducing effects for both K1 and the probiotic cell walls, but also revealed an even stronger effect when the two ingredients were blended. The growth inhibition of the blend reached a high level of statistical significance when compared to each ingredient alone. This particular data is of relevance for clinical pilot work currently being initiated in a small study of breast cancer patients.

The results in FIG. 18 demonstrate that K1, in itself a multifaceted ingredient with complex immune activating, anti-inflammatory, and effector-enhancing activities, exerts effects of significantly higher potency when blended with probiotic cell walls and metabolites. The results showed further evidence of the superiority of K1 above AHCC, both in terms of immune cell activation and induction of cytokines.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of any one of the aspects is applicable to all aspects and embodiments identified herein. Moreover, any of the features of an embodiment any one of the aspects is independently combinable, partly or wholly with other embodiments described herein in any way, e.g., one, two, or three or more embodiments may be combinable in whole or in part. Further, any of the features of an embodiment of any one of the aspects may be made optional to other aspects or embodiments.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

1. A combination for activating immune cells and/or inducing cytokines, the combination comprising:
a bioactive fungal extract derived from one or more different strains of Lentinula edodes, wherein the bioactive fungal extract comprises about 60%-90% by weight carbohydrates, comprising polysaccharides including α(1-4) glucans and β(1-3) glucans; and
a probiotic component,
wherein the bioactive fungal extract and the probiotic component interact synergistically to activate immune cells and/or induce cytokines.

2. The combination of claim 1, wherein the bioactive fungal extract is K1.

3. The combination of claim 1, wherein the probiotic component is selected from the group consisting of one or more Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

4. The combination of claim 1, wherein the probiotic component is B. lactis or metabolites and/or probiotic cell wall fractions derived from cultures of B. lactis.

5. The combination of claim 1, wherein the bioactive fungal extract and the probiotic component are formulated together in a dosage form for oral delivery.

6. The combination of claim 5, wherein the dosage form for oral delivery is selected from tablets, capsules, liquids, chewables, soft gels, sachets, powders, syrups, liquid suspensions, emulsions or solutions.

7. The combination of claim 1 for treating and/or preventing cancer, autoimmune diseases, allergies and inflammation, and/or an infectious disease.

8. (canceled)

9. (canceled)

10. A method for activating immune cells, the method comprising:
administering to the immune cells a combination comprising an effective amount of a bioactive fungal extract and an effective amount of a probiotic component,
wherein the effective amounts are sufficient to synergistically activate the immune cells.

11. The method of claim 10, wherein the bioactive fungal extract is K1.

12. The method of claim 10, wherein the probiotic component is selected from the group consisting of Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

13. The method of claim 10, wherein the probiotic component is B. lactis or metabolites and/or probiotic cell wall fractions derived from cultures of B. lactis.

14. The method of claim 10, wherein the bioactive fungal extract and a probiotic component in the combination are administered simultaneously and/or sequentially.

15. (canceled)

16. The method of claim 10, wherein the immune cell activation includes upregulating expression of CD69 on lymphocytes and monocytes, activation of one or more of NK cells, monocytes and macrophages, activation of one or more of anti-inflammatory cytokines IL-1ra and IL-10, and/or activation of one or more of IL-8, G-CSF and TNF-alpha.

17. (canceled)

18. (canceled)

19. (canceled)

20. A method for reducing viability of cancer cells, the method comprising:
administering to the cancer cells a combination comprising an effective amount of a bioactive fungal extract and an effective amount of a probiotic component,
wherein the effective amounts are sufficient to synergistically reduce the viability of the cancer cells.

21. The method of claim 20, wherein the bioactive fungal extract is K1.

22. The method of claim 20, wherein the probiotic component is selected from the group consisting of Lactobacillus plantarum, Lactobacillus acidophilus, Lactobacillus paracasei, Leuconostoc mesenteroides, Lactobacillus bulgaricus, Lactobacillus sasei, Lactobacillus salivarius, Pediococcus pentosaceus, Streptococcus thermophiles, Bacillus subtilis, Bacillus coagulans, Enteroccous faecium, Bifidobacterium bifidum, Bifidobacterium lactis (B. lactis), Bifidobacterium longum, Lactobacillus rhamnosus, Lactobacillus reuteri, and Bifidobacterium infantis.

23. The method of claim 20, wherein the probiotic component is B. lactis or metabolites and/or probiotic cell wall fractions derived from cultures of B. lactis.

24. The method of claim 20, wherein the bioactive fungal extract and a probiotic component in the combination are administered simultaneously and/or sequentially.

25. (canceled)

26. A kit for activating immune cell function, the kit comprising:
a combination of an effective amount of a bioactive fungal extract, selected from AHCC or K1, and an effective amount of a probiotic; and
instructional materials for using the combination to treat or prevent a condition for which activating immune cell function may be therapeutic or prophylatic;
wherein the amounts of bioactive fungal extract and probiotic are effective in combination to synergistically active immune cell function.

27. The kit of claim 26, wherein the bioactive fungal extract is K1.

28. The kit of claim 27, wherein the synergistic activation of immune cell function comprises one or more of the following:
upregulating expression of CD69 on lymphocytes and monocytes;
activation of one or more of NK cells, monocytes and macrophages;
activation of one or more of anti-inflammatory cytokines IL-1ra and IL-10;
activation of one or more of IL-8, G-CSF and TNF-alpha; and
synergistic reduction cancer cell viability.
